(19) United States
(12) Patent Application Publication
(10) Pub. No.: US 2019/0223747 A1
(43) Pub. Date: Jul. 25, 2019

(54) WEARABLE PHYSIOLOGICAL ACTIVITY SENSOR, SENSING DEVICE, AND SENSING SYSTEM

(71) Applicant: Chang-an CHOU, Taipei (TW)

(72) Inventor: Chang-an CHOU, Taipei (TW)

(21) Appl. No.: 16/070,528

(22) PCT Filed: Jan. 20, 2017
(86) PCT No.: PCT/CN2017/071978

§ 371 Date: Jul. 17, 2018

(30) Foreign Application Priority Data
Jan. 22, 2016 (CN) 201610044155.4
May 30, 2016 (CN) 201610374046.9
Jan. 18, 2017 (CN) 201710039180.8
Jan. 18, 2017 (CN) 201710040021.X
Jan. 18, 2017 (CN) 201710040024.3

(51) Int. Cl. A61B 5/0478; A61B 5/00; A61B 5/04

(52) U.S. Cl. CPC A61B 5/0478; A61B 5/681; A61B 5/04012; A61B 5/0205; A61B 5/6817; A61B 5/4812; A61B 5/6816

(57) ABSTRACT

The present invention is related to a wearable physiological activity sensor, sensing device and sensing system, which employs at least an ear-worn structure to install physiological sensing element(s), thereby acquiring physiological signals from the head and/or the ear(s) of a user. The sensor, sensing device and sensing system also can be used to perform procedure(s) capable of influencing the user's physiological state in accordance with the physiological signals.

### FIELD OF THE INVENTION

The present invention is related to a wearable physiological activity sensor, sensing device, and sensing system, and more particularly, related to a wearable physiological activity sensor, sensing device, and sensing system which employs at least an ear-worn structure to install physiological sensing element(s), so as to achieve the acquisition of physiological signals.

### BACKGROUND OF THE INVENTION

Traditionally, brain electrical activities measured through electrodes placed on the head are known as the electroencephalogram (EEG). EEG can be used for detecting and diagnosing various physiological conditions, and the brain electrical activity information obtained can also have other applications, such as learning concentration level, fatigue level, brain computer interface (BCI) and so on.

In general, the measurement methods of brain electrical activities can be divided into two types, reference montage and bipolar montage. In the reference montage, the brain electrical activity of one identical location is used as reference. For example, commonly, the reference electrode is placed at a location where there is no brain cortex electrical activity, and the activity detection electrodes acquire the brainwaves (EEG signals) relative to the reference electrode. In the bipolar montage, the brain electrical activity potential differences between two locations are measured as the brainwaves.

Nevertheless, traditional brain electrical activity monitoring devices are typical of the drawbacks of heavy, complicated wiring and requiring professionals for the placement of electrodes such that they cannot be popularized with ease. Accordingly, to overcome such drawbacks, various types of improvements have been developed, and one of the improvements is the ear-worn brain activity monitoring device.

For example, Looney D, et al., “The in-the-ear recording concept: user-centered and wearable brain monitoring.” IEEE PULSE, 2012 November-December; 3(6):32-42. describes the method of obtaining EEG signals via the ear canal, and it also proves that EEG signals acquired via the ear canal shows similar waveform changes as EEG signals acquired from the temporal lobe. In addition, there are also numerous patents disclose different methods of using ear as the location for obtaining EEG signals. For example, US20070112277 discloses the usage of ear canal inner plug as a medium for the installation of EEG electrodes; US20120209101 discloses the usage of a hearing aid matching with the shape of auricle as a medium for installing EEG electrodes; U.S. Pat. No. 8,565,852 discloses the method of using an ear hooking structure along with a clamp to achieve the effect of electrode securement; US20060094974 describes the concept of using the structure of auricle to install electrodes; and U.S. Pat. Nos. 7,197,350 and 8,781,570 disclose the usage of earmuffs as a medium for the installation of electrodes.

However, since the space inside the ear canal is extremely small, the electrode cannot be positioned easily and accordingly the manufacturing of the monitoring device becomes complicated, such that the implementation of the device is relatively difficult. In addition, there is another issue associated with the sampling inside the ear canal, and it is the earwax. Earwax inside ear canals is a substance that is naturally generated by the human body, and it might reduce the contact between the electrode and the skin of the ear canal, or it might even completely isolate the contact therebetween, such that excellent contact between the electrode and the skin might not be achieved easily. Consequently, before wearing the device each time, the cleaning procedure is required, which can be an extremely tedious process to users.

Furthermore, when the installation location of the electrode is at the connecting area between the auricle and the skull, since such area is a plane closely attached to the skull, in order to maintain the contact of the electrode with such plane, it is necessary to apply a force toward the direction of the skull on the electrode. However, within this area of auricle, there is no structure available that can be relied thereon for applying force toward this direction. As a result, the question on how to secure the electrode is always an issue to manufacturers seeking solutions thereof. Moreover, it is also necessary to consider the use comfortableness that should not be scarified while maintaining the stable contact of the electrode.

For example, in US 2006/0094974, it utilizes the common clamping method for securing the reference electrode onto the earlobe, and the detection electrode is secured by utilizing the physiological structure of the auricle. Such type of method seems to be a nice try; however, since there is no securing force on the detection electrode, in fact, the contact between the electrode and the skin is extremely unstable, and the electrode moves along with the rotation or movement of the head. Therefore, the quality of the signals acquired is affected directly.

Furthermore, in U.S. Pat. No. 8,565,852, it discloses that to secure the detection electrode at a space among the triangular fossa, the crus of helix and the superior crus of anthelix as well as to allow the electrode to contact the area attached to the skull in such space, a clamp with a special shape is used. Nevertheless, the clamping force might cause discomfort on the user after using a long period of time. Furthermore, in this patent, it also discloses another method of using an ear hooking structure for maintaining the detection electrode at a desired contact location. However, it is found that such method cannot provide a force directly applied onto the electrode; therefore, the electrode can still be moved easily, and thus cannot be maintained to stably contact with the skin for a long period of time. Consequently, the quality of acquired signal might be reduced.

Regarding the disclosure of US2012/0209101, despite that it uses an ear-shaped hearing aid to carry the electrode and to ensure the contact of the electrode with the ear canal and the auricle skin, nevertheless, in such method, the force of securement mainly comes from the frictional force exerted between a portion of the device entering into the ear canal and the ear canal; in addition, the shape of the hearing aid and the hooking piece extended to the rear of the ear are for the purpose of positioning only. Consequently, the electrode outside the ear canal lacks a direct securement force. As a result, if the portion of the device entering into the ear canal slips thereinside, the electrode is very likely to disengage from the skin surface of the auricle. Therefore, unstable electrode contact can still occur easily.

In addition, in US20070112277, it not only discloses the embodiment related to the installation of electrode inside the ear canal, but also discloses the method of placing the electrode at the surface of a housing behind the ear so as to contact the skull. This is a very common installation method and contact location for ear-worn brain activity monitoring devices. However, for this kind of structure, it is not easy for the housing behind the ear to generate a force toward the direction of the skull, so that, in general, the housing behind the ear is merely maintained behind the ear. Consequently, the device can be moved easily, and the contact between the electrode and the skill is not stable.

Recently, the 3D scanning method is further developed and utilized to allow each individual user to have an in-ear device that completely matches his or her own ear shape. For example, US20150168996 discloses the use of 3D scanning technology to form a device matching with the user's ear shape for the installation of the sensor. In addition, United Sciences, LLC even provides the field service for ear profile scanning. The purpose of such tedious and resource-consuming process is to ensure that the physiological sensor can be stably installed inside an ear without being affected by the movement of the head, such that high-quality signals can be obtained.

In view of the above, it can be understood that currently, in the field of ear-worn devices equipped with physiological sensors, manufacturers are still seeking solutions for installing physiological sensors with greater stability. Therefore, how to overcome the above-mentioned various drawbacks is indeed an important issue for the current ear-worn brain activity monitoring device.

### SUMMARY OF THE INVENTION

During the process of finding the solutions, beyond the existed positions, the applicant discovers a novel position also capable of being used to acquire EEG signals, namely, the auricle which is protruded out of the skull and mainly formed of cartilage covered by skin, and after experiments, it is known that the strength of EEG signals acquired on the auricle is sufficient to perform the relative analyses and provide the information about brain activity.

Therefore, the object of the present invention is to provide an ear-worn brain activity sensor, which employs an in-ear housing having a size and shape at least partially matching with the cymba conchae and/or the cavum conchae of an auricle of an user, so that the activity detection electrode thereon can have a stable contact with the concha wall of the auricle, thereby facilitating the acquisition of EEG signals from the area around temporal lobe.

Another object of the present invention is to provide an ear-worn brain activity sensor, which employs an in-ear housing having a size and shape at least partially matching with the cavum conchae and/or the intertragic notch of an auricle of an user, so that the reference electrode thereon can have a stable contact with the tragus and/or the intertragic notch of the auricle, thereby facilitating the acquisition of EEG signals together with the activity detection electrode.

Another object of the present invention is to provide an ear-worn brain activity sensor which is engaged on the auricle through an interactive force between a front ear member and an extension member, so that the activity detection electrode or reference electrode located on the extension member can have a stable contact with the skin at the convex side of the auricle, thereby facilitating the acquisition of EEG signals.

Further another object of the present invention is to provide an eyeglass type brain activity sensor which employs an eyeglass structure to achieve a stable contact between the electrode(s) thereon and the skin at the convex side of the auricle and/or near the ear, thereby facilitating the acquisition of EEG signals.

Further another object of the present invention is to provide a brain activity sensor which includes a light emitting element and a light receiving element for acquiring physiological information about heart rate and/or oxygen saturation, so as to being a basis of biofeedback and/or breath training.

Further another object of the present invention is to provide a brain activity sensor which further includes ECG electrodes for acquiring ECG signals, and thus information about heart activity.

Still another object of the present invention is to provide an ear-worn electrode structure, which employs an elastic material for achieving a stable contact between the electrode (s) and the ear canal.

Still another object of the present invention is to provide a brain activity sensing device which is combined with an earphone for being integrated into the user's daily life more.

Still another object of the present invention is to provide a brain activity sensing device which includes a wearable structure capable of engaging with the neck or the head, so as to provide plural usages.

Still further another object of the present invention is to provide a wearable electrical stimulation device, which is mounted on the user through an eyeglass structure or an ear-worn structure, thereby providing the portability.

### BRIEF DESCRIPTION OF THE DRAWINGS

FIG. 1 is a schematic view showing the position of cerebral cortex within the skull and the position of auricle;

FIG. 2 is a comparison chart between EEG signals acquired by using the electrode installation method of the present invention and the known scalp electrode installation method;

FIG. 3 is a schematic view of the concave side of an auricle;

FIGS. 4a-4c are schematic views showing in-ear housings in preferred embodiments of the present invention, and the engagements thereof with the inner side of auricle;

FIGS. 5a-5b illustrate one in-ear housing how to adapt to different sizes of auricles in a preferred embodiment of the present invention;

FIGS. 6a-6b illustrate the electrode locating at a position of the in-ear housing capable of contacting the concha floor in preferred embodiments of the present invention;

FIGS. 7a-7e, 8a-8c, 9 illustrate examples of the present invention for installing electrode(s) within the ear canal in preferred embodiments of the present invention;

FIGS. 10a-10d, 11a-11d, 12, 13a-13d illustrate examples of electrode contact assurance structure on the in-ear housing in preferred embodiments of the present invention;

FIGS. 14a-14d are schematic views showing ear-hook structures in preferred embodiments of the present invention and the engagement thereof with the auricle;

FIG. 15 shows the enlarged view of a V-shaped recess between the auricle and the skull;

FIGS. 16a-16c illustrate examples for mounting electrode(s) on the in-ear housing in preferred embodiments of the present invention;

FIGS. 17, 18a-18d, 19a-19e, 20 illustrate examples for mounting electrode(s) on the ear hook structure in preferred embodiments of the present invention;

FIG. 21 is a schematic view showing the in-ear housing having electrodes as well as light emitting element and light receiving element mounted thereon in a preferred embodiment of the present invention;

FIGS. 22a-22f, 23a-23e are schematic views showing an eyeglass structure with electrode(s) mounted thereon in preferred embodiments of the present invention;

FIGS. 24a-24c illustrate examples of a wearable structure capable of mounting on the head and on the neck in preferred embodiments of the present invention;

FIGS. 25a-25b illustrate examples of a wrist-worn brain activity sensing device in preferred embodiments of the present invention;

FIGS. 26a-26c are schematic views showing the brain activity sensing device with connection structure in preferred embodiments of the present invention;

FIGS. 27a-27c are schematic views showing attaching element(s) in preferred embodiments of the present invention; and

FIGS. 28a-28b illustrate the combination of ear-worn structures and head-mount structure in preferred embodiments of the present invention.

### DETAILED DESCRIPTION OF THE INVENTION

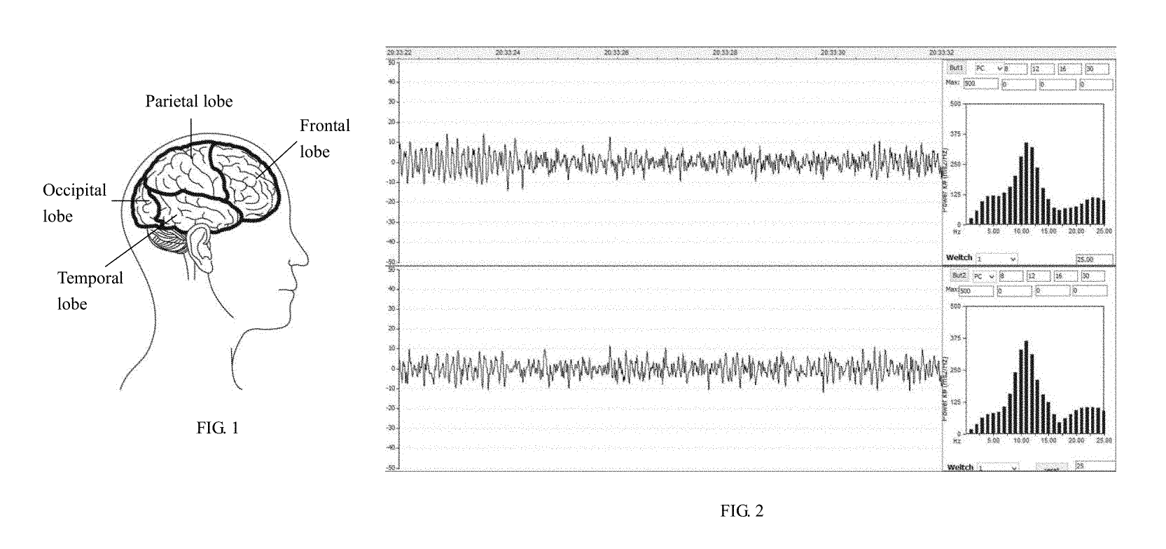

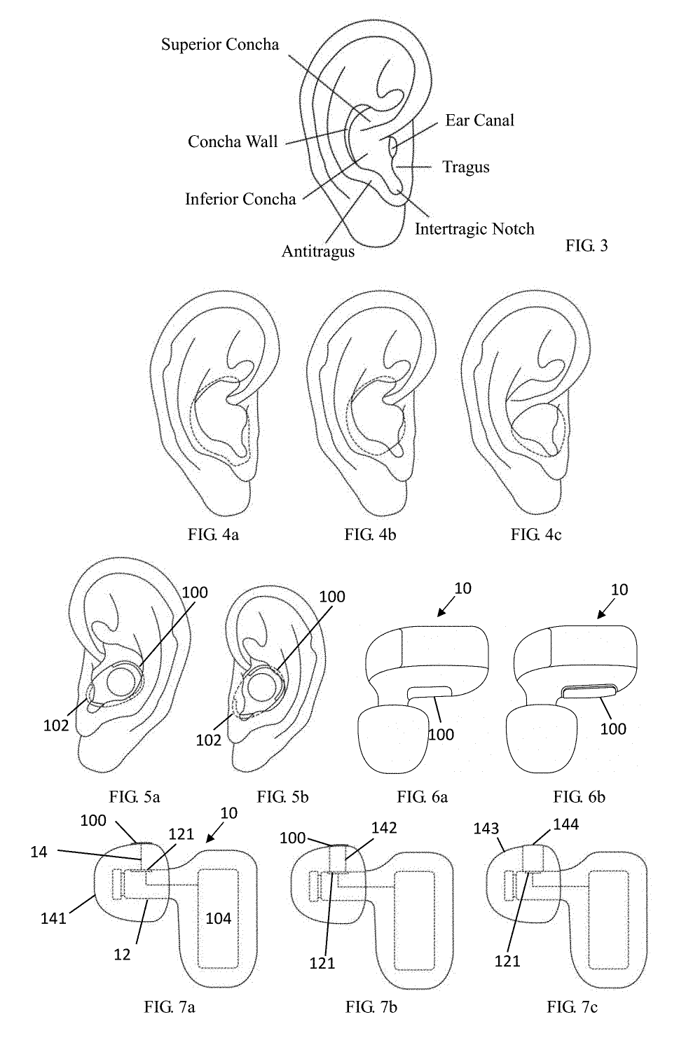

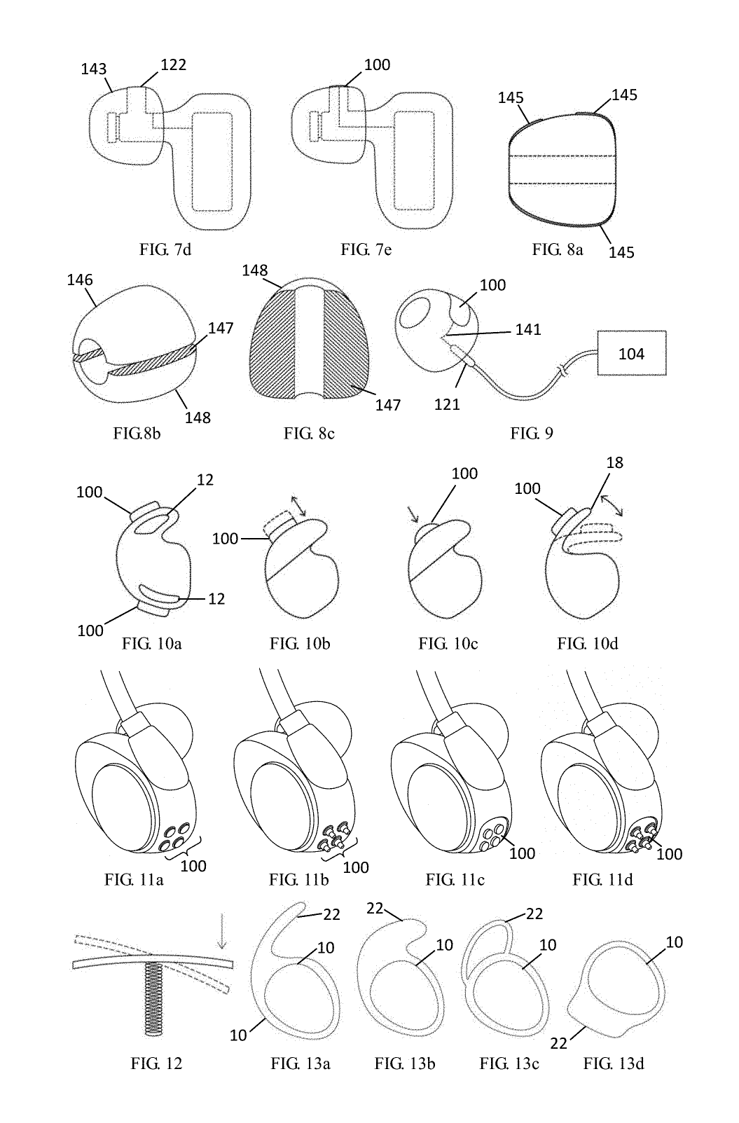

First, please refer to FIG. 1, a schematic view showing the location of the cerebral cortex in the skull and the locations of the auricles. As shown, it can be seen that the cerebral cortex is at the upper half of the skull, and the auricles (also known as “pinna”) are located at the two sides of the skull and extruded out of the skull. And, generally, the cerebral cortex is located between the upper halves of the auricles, which are separated by the ear canals.

Experimental results have indicated that excellent EEG signals can be acquired from the upper portions of the auricles, and the EEG signals become weaker as the measurement location moves downward. After observing the physiological structure of the head, the explanation should be the location inside the skull that the upper halves of the auricles are corresponding thereto is the cerebral cortex; therefore, under such condition, EEG signals can be acquired at the upper halves of the auricles through the transmission of the skull and the aural cartilages. On the other hand, since the lower halves of the auricles are at a distance further away from the cerebral cortex and due to the additional separation of the ear canal, EEG signals become weaker as the location moves further away downward. Accordingly, in the present invention, in principle, the ear canals are used as the boundary. The upper halves of the auricles can be regarded as the locations capable of obtaining EEG signals and suitable for the installation of activity detection electrodes, and the lower halves of the auricles are considered as the locations with relatively weak EEG signals and suitable for the installation of reference electrodes.

Furthermore, it shall be particularly noted that there is a location which is very suitable for installing the reference electrode, that is, the tragus. Tragus, physiologically, is part of the auricle, and the location inside the skull that the tragus is corresponding thereto is not the cerebral cortex. Moreover, in experiments, it is relatively rare to detect EEG signals at tragus. And, the structure of tragus is relatively independent. Consequently, tragus is a location that is particularly suitable for the installation of reference electrode.

Please refer to FIG. 2, a comparison chart between EEG signals acquired by using the electrode installation method of the present invention and the known scalp electrode installation method. The top drawing refers to EEG signals acquired with the activity detection electrode installed on the scalp at top of the auricles (i.e. in the traditional 10-20 system, the location of T7/T8) along with the reference electrode installed on the earlobe. The bottom drawing refers to EEG signals acquired with the activity detection electrode installed on the upper half of the auricle at the same side along with the reference electrode installed on the tragus.

From the drawings, it can seen that both show the same change pattern; therefore, it can be understood that when the activity detection electrode is installed on the upper half of the auricle, it is similar to installing the activity detection electrode on the scalp, both can acquire EEG signals of the temporal lobe.

In the following, details on how to use such novel EEG electrode contact locations to achieve the effect of overcoming the drawbacks of the prior arts are described.

Please refer to FIG. 3, showing a schematic view of the inner side of the auricle. Auricle is the part of the ear protruding out of the skull, and it is mainly formed of cartilage covered by skin, and the lowest portion thereof is the lobe (also known as “lobue”) which only comprises subcutaneous tissues. The inner side (concave side) of the auricle includes various bulges and recesses as shown in the drawing.

According to the concept of the present invention, in the structure of auricle, skin surfaces with cartilage thereunder, such as the backside (convex side) of the auricle, and the inner side thereof, all can be the installation and contact locations for EEG electrode. Here, due to being protruding out of the skull, the auricles are suitable for hanging or fixing, and further, as shown in FIG. 3, the bulges and recesses at the inner side of the auricles are also suitable for installing and securing electrodes. Accordingly, in cooperation with the above-mentioned novel sampling locations of the present invention, it will be able to provide a securing method that is easier to achieve stable electrode contact.

For example, at the inner side of the auricle, the surroundings of the superior concha and the inferior concha include a vertical planar area extended from the concha floor (i.e. the plane parallel to the skull) upward to connect to the antihelix and the antitragus, which is known as the concha wall. This natural physiological structure of the ear provides a continuous vertical plane protruding out of the concha floor; therefore, when such area is used as the electrode contact area, the force required for securing the electrode can be in the radial direction, i.e., the direction parallel to the concha floor, which is different from that adopted in the prior arts. Furthermore, the intertragic notch, which is directly adjacent to the concha wall and located between the antitragus and the tragus, as well as the tragus also provide a contact area protruding out of the concha floor. Accordingly, in the present invention, the continuous planar area formed by the concha wall, the antitragus, the intertragic notch and the tragus is particularly suitable for the installation of electrode, in which it allows to use the radial force to achieve the stable contact. Therefore, the drawback of the prior arts that there is difficulty in providing a stable maintaining force on the electrode toward the concha floor can be overcome.

In addition, since the scope of such vertical planar area extends from the upper part of the auricle to the lower part of the ear canal, based on the experimental results mentioned above, the area above the ear canal can be used as the contact location for the activity detection electrode, such as the concha wall located above the ear canal, whereas the area below the ear canal can be used as the contact location for the reference electrode, such as the concha wall located below the ear canal, the concha wall adjacent to the antitragus, the antitragus, the intertragic notch and the tragus.

The advantage is that within the confined space of one single ear, the installations of reference electrode and/or activity detection electrode can be completed, so that it is able to effectively utilize the reference montage to acquire EEG signals without being bound by the limitations of prior arts, namely, typically, the reference electrode should be installed at the mastoid or clamped on the lobe, and the activity detection electrode should be installed on the skull where corresponds to the location of cerebral cortex. As a result, for wearable physiological monitoring devices, this is definitely a major breakthrough in terms of feasibility and operation convenience, since the volume can be minimized and the wiring complexity also can be simplified, thereby providing the user the better using experience.

Here, it shall be noted that since rather than right-angled changes, the inner side of the auricle has smooth curve profile changes among the budges and recesses in terms of its actual physiological structure, there is no obvious right-angled boundary between the aforementioned vertical planar area and the concha wall, and generally, the two are connected via a curved change. Consequently, under such condition, the contact location of the electrode, except the vertical planar area, can also be the curved change depending on the structure differences of electrodes that are used for achieving contact, without limitation.

Furthermore, it shall also be noted that during the measurement of EEG signals, in addition to the reference electrode and the activity detection electrode, ground electrode is also frequently installed in order to achieve the suppression of common noises. However, in some circuit designs, the ground electrode also can be exempted depending on the actual demands. Therefore, for the purpose of being concise, the descriptions related to the ground electrode are omitted. Nevertheless, it can be understood that for the brain activity sensor and sensing device of the present invention, it also can be selected to install the ground electrode or not depending on the practical needs without limitation.

When the main objective is to contact such vertical planar area, an in-hear housing for installing at the inner side of the auricle is the priority choice. Regarding the type of shape and form of such housing, there are no particular limitations, and as long as the housing is able to achieve stable contact with the vertical planar area. For example, FIGS. 4a-4c are schematic views showing in-ear housings in preferred embodiments of the present invention that are located in the inner side of auricle, which respectively show the situations where the in-ear housing 10 is in contact with the entire portion, upper half portion and lower half portion of the vertical planar area formed by the concha wall, the antitragus, the intertragic notch and/or the tragus.

Particularly, in the present invention, the in-ear housing is preferably secured through the radial forces that are rejecting against the surrounding structure of the cymba conchae and/or the cavum conchae. Since the electrode contact location—the concha wall, the antitragus, the intertragic notch and/or the tragus—is at the surrounding of the cymba conchae and/or the cavum conchae, the effect of stabilizing the electrode contact can achieved while securing the in-ear housing.

One of the embodiments is to form the shape of the in-ear housing to match with the cymba conchae and the cavum conchae. Under such condition, the electrode is able to contact with the predefined location easily and the installation is facilitated the most. Another embodiment is to use a specially designed shape of in-ear housing in order to allow it to adapt to different auricle shapes and sizes of different users through simple operations. For example, as shown in FIGS. 5a-5b, the in-ear housing is configured to adapt to different sizes of auricles through simple rotating movements and to achieve the securement via rejecting. Under such condition, an electrode 102 can be installed at a location in contact with the tragus as the reference electrode, and another electrode 100 can be installed on the in-ear housing at a location relatively remote from the tragus contacting location, or can be installed at the location of the in-ear housing facing toward the concha floor (as shown in FIGS. 6a-6b) as the activity detection electrode. Accordingly, the contacts of the electrodes can be achieved while securing the electrodes. Since the electrode contact location of the in-ear housing might not be exactly the same in different auricles (as shown in FIGS. 5a-5b), preferably, the electrode 10 is formed to be a continuous surface capable of covering a relatively larger movement range in order to ensure the achievement of the contact.

In view of the above, it can be understood that the concept of the present invention is applicable to common types of earphones available in the market. As it is known, when a conventional earphone is installed at the auricle inner side, it is able to at least contact with the locations of the tragus, the intertragic notch or the antitragus naturally; furthermore, depending on its actual shape, whether it contacts with the concha wall is further determined. Consequently, the electrode can be installed at these locations. Moreover, when an earphone includes a portion extended into the ear canal, then the securement effect can be enhanced, and thus facilitating the stabilization at the inner side of auricle.

As a result, when implementing the in-ear housing of the present invention, there can be similar choices. The securement can be achieved through only the radial rejecting between the housing and the vertical planar area, or it can further include a portion entering into the ear canal in order to enhance the securement effect. In addition, the portion entering into the ear canal can also be provided with the sound function in order to guide sounds into the ear canal.

In addition that both electrodes are configured to contact with the aforementioned vertical planar area, it can also be configured in such a way that one of the electrodes is in contact with other location. For example, when the in-ear housing is configured to include a portion entering into the ear canal, then the electrodes can be installed respectively at a location capable of contacting with the tragus and at a location opposite to the tragus and separated by the ear canal, i.e. the corning area connected to the concha floor and the ear canal, as shown in FIG. 6a. In such condition, similarly, it also can be able to use the radial force to stabilize the contact. Furthermore, the advantage of such contact location is that as long as the portion entering into the ear canal can be firmly installed, the installation of electrode can then be completed, such that it is not only convenient but also extremely simple for use and installation.

In another preferred embodiment, one of the electrodes can be installed at a location in contact with the concha floor, as shown in FIG. 6b. Under such condition, since the in-ear housing has utilized the radial force to allow one of the electrode to form stable contact with the vertical planar area and thus being secured inside the auricle, the possible relative movement between the in-ear housing and the ear has been minimized. As a result, the location facing toward the concha floor where the electrode is mounted can be secured to a certain extent and thus has reduced movement. Therefore, this is also an advantageous contact method. For example, in the embodiments of in-ear housings as shown in FIGS. 4a-4C and FIG. 6a, one of the electrodes thereof can be installed on the surface in contact with the concha floor.

In still another preferred embodiment, an electrode can be installed on a surface of the portion entering into the ear canal in order to contact with the ear canal, wherein the location contacting with the upward position of the ear canal can be used to mount the activity detection electrode, and the location contacting the downward position of the ear canal can be used to mount the reference electrode. Therefore, there are numerous possibilities.

Regarding how to mount the electrode(s) on the portion entering into the ear canal, there are various possible selections.

As shown in FIG. 7a, the in-ear housing can be configured to have a supporting body 12 extended outward, and an elastic member 14 can be mounted on the supporting body. With the elastic restoring force of the elastic member, it can be compressed to facilitate the installation inside the ear canal. And, after entering into the ear canal, the elastic restoring force allows it to be firmly maintained inside the ear canal. Furthermore, if it is equipped with the earphone function, the supporting body can be configured to have a hollow channel in order to allow sounds to be transmitted into the ear.

Accordingly, as mounting the electrode, it is preferable to mount on a surface of the elastic member 14; therefore, not only the electrode can enter into the ear canal easily, but also the elastic restoring force of the elastic member can be utilized to naturally and firmly contact the electrode with the ear canal, which is advantageous.

As for the mounting of electrode, there are various possibilities.

For example, as shown in FIGS. 7a-7b, an electrode, such as thin metal or conductive fiber, can be attached onto a surface of the elastic member. Under such condition, it is necessary to consider how to electrically connect the electrode 100 on the elastic member surface to the circuit 104 inside the in-ear housing. In a preferred embodiment, the surface of the supporting body 12 can be configured to include a conductive portion 121 so as to achieve the connection between the electrode 100 and the circuit 104 through the conductive portion. For example, as shown in FIG. 7a, connecting wires can be used to connect the electrode 100 to the conductive portion 121 and connect the conductive portion 121 to the circuit 104. Alternatively, a different connection method also can be used between the conductive portion 121 and the electrode 100. For example, as shown in FIG. 7b, a conductive object 142 can be arranged between the two and in contact with the two at the same time such that the effect of electrical connection can still be achieved. Such method is more advantageous to maintain the contact between the electrode and the ear canal. It shall be noted that although only one single electrode is illustrated in the drawings, it can also be configured to include more than one electrode, without limitation.

For such configuration, there is one special embodiment, in which the electrode and the conductive object can be made of one identical conductive material, i.e. two are integrally formed as one single piece. Accordingly, as shown in FIG. 7c, it is equivalent to that the elastic member is formed by two types of materials, the elastic material portion 143 and the conductive material portion 144. Here, the portion formed by the conductive material can be used as the electrode and the conductive portion at the same time; whereas the portion formed by the elastic material can be used as the main body of the elastic member for providing the elastic restoring force in order to ensure the contact between the conductive material portion 144 and the ear canal. Under such condition, if the conductive material is also equipped with the elasticity, such as elastic conductive rubber, elastic conductive silicone, elastic conductive foam, then the elastic member is of elasticity as a whole.

Furthermore, in another preferred embodiment, the supporting body can be configured to be made of a conductive material directly. Therefore, the supporting body as a whole can be regarded as the conductive portion, further simplifying the implementation of the device.

Accordingly, in a preferred embodiment, as shown in FIG. 7d, the supporting body can be directly formed to include a protrusion 122 in order to replace the aforementioned conductive material portion. Consequently, when it enters into the ear canal, the exposed surface of the protrusion can be regarded as an electrode for contacting the ear canal. Through such method, since the elastic member is mostly made of an elastic material except the small area of the protrusion, the elastic restoration fore can still ensure the stable contact between the protrusion and the ear canal. In addition, as long as the area of the protrusion is appropriate, even if it is made a relatively rigid material, users may still not feel any discomfort. Alternatively, it can also be configured as shown in FIG. 7e in such a way that the electrode 100 is mounted on the exposed surface on the protrusion without utilizing the conductive material to form the supporting body. Therefore, it can be different kinds, without limitation.

In another embodiment, it is configured to directly use the elastic member made of a conductive material, such as conductive rubber, conductive silicon and conductive foam. Therefore, since the supporting body is made of a conductive material, then it only needs to be further connected to the circuit. Alternatively, the supporting body can be equipped with a specific conductive portion, and then, it only requires further confirmation that the elastic conductive portion contacts stably with the conductive portion. Consequently, regardless of any methods, the device is quite convenient to users. Furthermore, the outer surface can be further covered with a conductive fiber such that it is able to provide greater comfortableness for the contact with the skin, and it can also increase the useful lifetime, for example, the material of rubber and foam may have surface shedding due to frequent uses. Such design is particularly suitable for EEG measurement which employs electrodes installed at two ears. When EEG signals acquisition is performed via two ears, because the distance therebetween is sufficient, the contact locations of the electrodes are not limited, and even if the entire surface of the elastic member is configured to be conductive, the acquirement of signals is still be unaffected.

In addition, if the electrode has a specific contact location, such as the location facing upward is used as the activity detection electrode or the location facing downward is used as the reference electrode, then the outer surface of the conductive elastic member can be further covered with a non-conductive material, as shown in FIG. 8a. The outer surface can be covered with an insulative coating layer 145, and the location for contact can be exposed in order to be used as the electrode. In such design, the elastic member is made of one type of material only such that there is no need to combine different materials; in addition, it only needs to include the additional step of covering of insulative layer. Consequently, it is not only easy for manufacturing and facilitated for implementation but also a method with competitive advantages.

In still another embodiment, as shown in FIG. 8b-8c, the conductive elastic member is configured to include two portions, a first portion 146 and a second portion 148. And, the two portions are electrically insulated from each other by an insulative portion 147. Therefore, it is equivalent to allowing one single elastic member to have two conductive areas insulated from each other. Consequently, when the device is used for measurement, one of the possibilities is to cover the outer surface with an insulative coating layer as well as to allow the first portion and the second portion to expose a first conductive area and a second conductive area respectively for being used as two electrodes. Another possibility can be that a further conductive object, such as metal conductive sheet or conductive fiber, is additionally arranged on the surfaces of the first portion and the second portion respectively in order to form the first conductive area and the second conductive area. In this embodiment, the first portion and the second portion are of the function similar to that of the aforementioned conductive object 142. Therefore, it can be changed according to the actual measurement requirements without limitation.

Furthermore, in another similar and feasible embodiment, the supporting body can be omitted, and accordingly, the comfortableness during usage can be improved. In this embodiment, as shown in FIG. 9, the electrode 100 at the surface of the elastic member is electrically connected to the conductive portion 121 via a connecting wire 141, which is then electrically connected to the circuit 104. In addition, the elastic members as shown in FIG. 7b, FIG. 7c, FIG. 8a and FIG. 8b also can be configured to omit the supporting body without limitation.

Furthermore, alternatively, under the condition where two in-ear housings are used, it can also be configured to contact each electrode with the concha floor of each ear. Since the in-hear housing is already secured by the radial force between the in-ear housing and the auricle, the electrode facing toward the concha floor can then achieve a stable contact with the skin, which is convenient no matter for implementation or operation. In addition, when the two electrodes are installed on two ears respectively, in comparison to the installation of two electrodes on one single in-ear housing, the contact locations for acquiring EEG signals are of relatively less limitation such that the operation is facilitated.

As to how to achieve the rejecting between the electrode and the ear so as to ensure electrode contact, there are various feasible methods. For example, it can be achieved via the selection of the material of the in-ear housing. In one instance, the in-ear housing can be made of an elastic material to have a size slightly greater than the range of the cymba conchae and/or cavum conchae, such that when the in-ear housing is placed into the ear, the elastic restoring force generated due to the compression of the elastic material can be utilized to achieve the rejecting effect.

When the in-ear housing is selected to be made of an elastic material, it can be implemented as the entire in-ear housing is made an elastic material, and the electrode is mounted at a specific location on the surface, such as a location capable of contacting with the vertical planar area. In addition, the elastic in-ear housing can also be formed to have a hollow portion, such that the compressibility and the deformation force thereof can be increased, and also, a portion of the circuit elements can be arranged inside the hollow portion. For example, when the device is configured to be equipped with the earphone function, a sound production element can be arranged inside the elastic in-ear housing.

Under such condition, similar to the aforementioned elastic member placed inside the ear canal, the surface can be formed of a conductive area for being used as the electrode, for example, the electrode can be mounted on the surface thereof or the electrode also can be formed by combining different materials, or alternatively, it can also directly use a conductive elastic material to form the in-ear housing, and then define the electrode contact location by covering the outer surface with an insulative layer. Furthermore, the number of electrode is not limited to be one, and it is also possible to have two electrodes at the same time; for example, one is for the activity detection electrode and the other is for the reference electrode, without limitation. In addition, as previously mentioned, when two in-ear housings are used, the contact location of the electrode is not limited, for example, the in-ear housing can be simply configured to be made of one single type of elastic conductive material; therefore, not only the acquirement of physiological signals but also the rejecting can be achieved, which is convenient.

Furthermore, the elastic in-hear housing is also suitable to comprise a portion entering into the ear canal, which means it can be equipped with a portion entering into the ear canal and, at the same time, a portion engaged with the bulge-recess structure outside the ear canal at the auricle inner side. Accordingly, in addition to achieving greater securement effect, there also have more selections of installing the electrode. For example, one electrode can be located on a portion entering into the ear canal, and another electrode can be located on the portion outside the ear canal, or both electrodes can be located on the portion outside the ear canal, or both electrodes can be located on the portion entering into the ear canal. There is no limitation.

Alternatively, it also can employ a contact assurance structure to allow the in-ear housing to generate a force exerted in the radial direction. For example, as shown in FIG. 10a, the in-ear housing can be configured to comprise a hollow portion 12 formed of an elastic material, and thus, the shape of the in-ear housing can expand and shrink arbitrarily along with the shape of space that the housing is placed therein, so as to adapt to different ear shapes of different users, such that the electrode 100 thereon can contact with the inner side of auricle firmly. Furthermore, the contact assurance structure can also be implemented in other forms, such as a spring mechanism, a button with a rebound force, and an extension member with elasticity, and the rejecting and securement effect still can be achieved. Moreover, particularly, the location of the rejecting can also be designed to be at a location where the electrode is located, thereby further ensuring the stability of the electrode contact. As shown in FIGS. 10b-10d, three types of electrode protrusions extending out of the surface of the in-ear housing and capable of shrinking as a force exerted thereon are disclosed. FIG. 10b shows a metal electrode 100 capable of shrinking independently and penetrating through the in-ear housing, such as a spring-loaded electrode, and a common type thereof is a pogo pin. FIG. 10c shows the configuration of an electrode 100 embedded at the surface of the in-ear housing and equipped with the restoring force as pressed. FIG. 10d shows an electrode 100 disposed on an extension member 18 with elasticity, which is able to adapt to the shape of the concha wall so as to provide a force for rejecting the electrode against the concha wall. Here, it can be the end portion of the extension member or the entire extension member to reject against the concha wall, without limitation. And, no matter which situation is employed, such configuration is able to facilitate the achievement of a more precise and stable contact between the electrode and the skin. Consequently, without limitation, as long as it matches with the ergonomic shape of ear and can generate the radial rejecting to secure the in-ear housing onto the cymba conchae and/or cavum conchae, such methods shall be within the scope of the present invention.

Alternatively, the contact assurance structure can also be implemented to be on the electrode 100 directly. For example, as shown in FIG. 11a, an electrode can be formed as a plurality of scattered contact points, such as parallel connected to each other, so that regardless which contact point is being contacted, it can be deemed that the contact between the electrode and the skin has been completed; therefore, it is very convenient to users. This is particularly useful to a contact surface with curvature or to the condition where tiny movements may occur. Furthermore, it is preferable that each scattered contact point can be configured to be of shrinking ability; for example, as shown in FIG. 11b, the pogo pin can be used in order to ensure the achievement of the contact. For example, the contact between the skin and the electrode can be achieved by using the compression generated by the pogo pins, so that movements of small distance occur between the skin and the electrode can be overcome by the shrinking ability of the pogo pins.

Furthermore, as shown in FIGS. 11c-11d, in another embodiment, one single electrode 100 can also be configured to include a plurality of protrusions thereon. For example, the electrode sheet can be directly configured to include a plurality of protrusions, or the electrode sheet can also be configured to include a plurality of shrinkable protrusions, without limitation. All of such configurations are able to facilitate the improvement of the contact between the skin and the electrode.

In addition, the electrode can also be implemented to be of a floating type. For example, as shown in FIG. 12, the shrinkable structure, such as pogo pin, can be arranged underneath the electrode. Accordingly, to cope with the change of the contact surface, the electrode is able to shrink in the vertical direction, and by utilizing the underneath pogo pin as pivot point, the electrode also can have angle changes, which is particularly useful in adapting to the shape of the auricle. Furthermore, the surface of the floating type electrode can also be formed with protrusions, such as the combination of the embodiments shown in FIGS. 11c-11d and FIG. 12, so that the contact can be achieved more easily.

It shall be noted that the above-described structures that for achieving the rejecting effect can be configured at any location of the in-ear housing, for example, it can be the locations in contact with the tragus, the antitragus, the concha floor, the concha wall, and/or the intertragic notch, and it is also not limited to be where the electrode is mounted. Moreover, more than two types of rejecting structure can be used at the same time in order to further ensure the achievement and maintenance of the contact. Consequently, it is not limited to any specific configurations.

In addition, according to different ear sizes of different users, the in-ear housing can also be configured to have different dimensions for users' selections. Alternatively, the overall dimensions of the in-ear housing also can be changed through exchanging covering members which cover the in-ear housing, such as silicon cover, thereby enhancing the cost effect. And, under such condition, preferably, the electrode is configured to penetrate out of the surface of the in-ear housing and be shrinkable as mentioned above. Accordingly, the change of the covering member would not affect the location of the electrode and the contact with the skin. Alternatively, the dimension of the housing also can be achieved by only changing a portion of the in-ear housing. For example, only a portion of the in-ear housing surrounding the shrinkable electrode is changed without replacing the electrode, which also is cost effective. Certainly, it can be understood that the part equipped with the electrode can also be configured to be replaceable. In addition, the material of the covering member can be further changed depending upon the needs, such as, silicon, rubber and foam are all excellent choices, and through selecting the material, the buffering effect can also be achieved, which is of great advantages. Consequently, it can be changed depending on the real demands without limitation and the present invention is not limited thereby.

Accordingly, in a preferred embodiment, the in-ear housing is implemented as shown in FIGS. 13a-13d. The in-ear housing 10 is able to adapt to different shapes and sizes of the inner sides of auricles through changing the covering member 20 equipped with the extension element 22. Since auricles have different sizes, the dimensions and shapes of the in-ear housing capable of inserted therein are different. Consequently, through the exchange of the covering member of different thickness, shapes and materials, the change of the extension element of different shapes, and/or the flexibility of the extension element, it is able to adapt to various types of auricles of different dimensions and shapes at the greatest possibility. It shall be noted that the electrode can also be configured to be unchanged while changing the covering member. For example, the aforementioned spring can be used for carrying the electrode thereon in order to overcome the thickness of the covering member; or the electrode can also be configured to mount directly on the covering member and electrically connect with a conductive contact portion of the housing when the covering member is covered onto the housing. Therefore, there is no limitation.

In the embodiment as shown in FIGS. 13a-13c, the extension element can reject against the concha wall on top of the cymba conchae and can be implemented to have various possible shapes. For example, the extension element as shown in FIG. 13a is thinner such that it is of a greater flexibility; whereas the extension element as shown in FIG. 13b has a greater width such that it is of greater supporting capability. Furthermore, it can also be formed to have a ring shape as shown in FIG. 13c. Therefore, there is no limitation.

Alternatively, the extension element can also be arranged at other locations. For example, as shown in FIG. 13d, the extension element is arranged at the lower portion of the housing, and it is able to achieve the rejecting against the concha wall below the cavum conchae (i.e. the location around the antitragus) via changing the thickness and shape thereof. Moreover, the extension element can also be arranged at the location in contact with the tragus, or it can be arranged at a location of the concha wall opposite to the tragus. As a result, through the arrangement of the extension element, the in-ear housing can be maintained at the auricle inner side in an even more stable state.

Particularly, in addition that the extension element is able to provide a radial rejecting force parallel with the concha floor, it can be further configured to have an inclination toward the concha floor. With such design, when the extension element is arranged at the auricle inner side, other than the locations of the concha wall, the tragus and the antitragus, it is able to further generate a component force toward the direction of the skull via the inclination, so as to stably maintain the in-ear housing on the surface of the auricle.

Furthermore, the extension element can also be configured to locate at the location of the in-ear housing facing toward the concha floor, such as an elastic protrusion facing toward of the concha floor, in order to achieve the contact with the concha floor. This is particularly suitable to the condition where the electrode contact with the concha floor.

Specifically, as shown in FIG. 3, in the physiological structure of the auricle, a bulge division is formed between the cymba conchae and the cavum conchae. When the aforementioned extension element is restricted by the concha wall at top of the cymba conchae, particularly when it has an inclination to provide a component force toward the skull, then the upper edge of the in-ear housing is able to naturally contact with such bulge division. Consequently, this is extremely helpful in achieving the contact between the electrode arranged at this location and the concha floor.

Furthermore, when the electrode is configured to be arranged on the covering member, it would be particularly suitable to be arranged on the extension element. Since the main purpose of the extension element is to form the rejecting against the vertical planar area, the arrangement of the electrode on the extension element is able to utilize the force of rejecting to achieve the stable contact between the electrode and the skin. For example, the extension element which contacts upwardly with the concha wall at top or the extension element which faces toward the concha floor can be relatively suitable for mounting the electrode.

In addition, without limitation, various different embodiments mentioned above can be integrated according to different contact locations of the electrode required so as to satisfy different implementation requirements. For example, when a single ear is used to obtain EEG signals, then the upward extension element (as shown in FIGS. 13a-13c) can cooperate with the downward extension element (as shown in FIG. 13d) for being secured at the auricle inner side. Under such condition, the installation location of the reference electrode can be chosen to contact with the tragus or the concha wall adjacent to the antitragus (via the downward extension element), and the installation location of the activity detection electrode can be chosen to contact with the concha wall at top of the cymba conchae or the concha floor; wherein the contact with the concha floor can be directly arranged on the surface of the in-ear housing, or can be achieved by utilizing the extension element facing toward the concha floor.

In an embodiment, when both ears are used to obtain EEG signals, since the distance therebetween is sufficient, the contact location of the electrode is not limited, so that it is mainly focused on allowing the in-ear housing to be stably maintained on the auricle inner side, and achieving a stable contact between the electrode and the skin. For example, it can be selected to contact the electrodes with the concha floors of both ears, or with the the concha wall, the antitragus or the tragus. Therefore, combinations can be made depending upon the actual needs, without limitation.

Particularly, in another embodiment, there are two in-ear housings and both are equipped with two electrodes of one reference electrode and one activity detection electrode. Then, through the two sets of reference electrodes and activity detection electrodes located on different ears respectively, two-channel EEG signals can be acquired. Alternatively, the two-channel EEG signals can also be acquired by installing only one reference electrode on one of the in-ear housings. Such embodiment can be used for, for example, monitoring the activities of left brain and right brain, which is also advantageous.

In view of the above, it can be understood that for stably maintaining the in-ear housing at the auricle inner side, at least two areas of rejecting shall be achieved primarily, for example, the securement force generated by the portion entering into the ear canal along with the rejecting force against the upper concha wall and/or the lower concha wall and/or the tragus from the portion outside of the ear canal; or the rejecting force against the upper concha wall as well as the rejecting force against the tragus and/or the lower concha wall from the portion outside of the ear canal. Therefore, during implementation, as long as it is a rejecting location capable of achieving the appropriate radial rejecting force, and as long as the contact between the electrode and the skin can be maintained, it shall be within the scope claimed by the present invention, and the present invention shall not be limited to the specific embodiments described above.

In another example, the backside (convex side) of auricle is also a location suitable for sampling. When such location is used as the location for sampling, the hook-typed structure would be the priority choice. In the present invention, different from the prior arts, the electrode located on the member or housing arranged at behind the ear is configured to contact with the backside of auricle, rather than the conventional contact location, the skull.

In general, a hook-typed device typically provides one member at the front of auricle and another member at the rear thereof, and the securement on the auricle is mostly achieved via the interaction between the two members. Therefore, it is relatively difficult to maintain the contact between the rear member and the skull. In comparison, the contact with the backside of auricle can be achieved easily, and such condition just meets the novel contact location proposed in the present invention.

As shown in FIGS. 14a and 14b, an ear-hooking structure according to a preferred embodiment of the present invention and the engagement between the ear-hooking structure and the auricle are illustrated. The ear-hooking structure shown in the drawings comprises a front ear member 60, preferably, as the in-hear housing as mentioned above, and an extension member 62 extended upward from the front ear member 60, cross over the top of the auricle, and reach the backside (convex side) of the auricle, wherein between the two members, there exist interactive forces for ensuring that the ear-hooking structure can be firmly maintained on the auricle. The electrode is mounted on the extension member at a location capable of contacting with the backside skin of the ear. Accordingly, the contact between the electrode and the skin can be stabilized naturally by the interactive forces between the front ear member and the extension member.

In this embodiment, similarly, when the contact location is at the upper portion of the auricle, it can be used as a sampling point for an activity detection electrode. If it is implemented as a reference electrode, then the contact location thereof can be located at the lower portion of the auricle. Further, the electrode also can be mounted on the front ear member to contact the inner side of the auricle, for example, the electrode on the in-ear housing can be configured to contact the upper half portion of the auricle inner side in order to be used as the activity detection electrode, or to contact the lower portion of the inner side of the auricle in order to be used as the reference electrode. In addition, the electrode also can be mounted on the portion entering into the ear canal in order to contact the ear canal. Therefore, different changes can be made according to the needs without limitations.

Regarding how to achieve the interactive between the front ear member and the extension member, there are also various different possibilities. For example, the structure can be designed to create a misalignment between the extension member and the front ear member in order to apply force on the ear naturally. Alternatively, a hinge structure can be employed to connect the two members, wherein the hinge axle can be configured to be parallel with (FIG. 14a) or perpendicular to (FIG. 14c) the concha floor in order to allow the extension member to generate a force toward the direction of the auricle backside. Alternatively, a sliding structure (FIG. 14d) also can be employed to connect the two members thereby allowing the extension member to obtain a force toward the auricle in a top-down manner.

Furthermore, the shape of the extension member can be designed to have a curvature matching with the backside of the auricle such that the stability of electrode contact can also be increased. Alternatively, the extension member can be made of an elastic material such that the elasticity of the material can be utilized to increase the contact stability of the electrode, for example, the elasticity thereof can interact with the front ear member to generate a force for clamping the auricle. Therefore, there are various possible embodiments, and the present invention is not limited to any specific configurations.

Accordingly, through the selection of appropriate extension member and appropriate interactive force application, demands of different electrode contact locations can be satisfied, for example, a location at the auricle backside corresponding to the concha wall of the auricle inner side, and a location at the auricle backside adjacent to the earlobe both can be easily contacted by the extension member to achieve stable contact, and further, the manufacturing and operation thereof also are convenient.

In addition to the aforementioned locations, there is still one location where can be contacted by the extension member in a stable and easy manner, and it refers to the V-shaped recess between the auricle and the skull, as shown in FIG. 15. The V-shaped recess is located between the auricle and the skull, and comprises a skull portion 901, an auricle portion 902 and a connecting portion 903 for connecting therebetween, which naturally form a physiological structure suitable for an object to be placed between the auricle and the skull. When an object is placed at such area, not only it can be selected to contact anyone of the three portions of 901-903, but also the auricle and the skull are able to naturally provide a force for clamping the object therebetween. Moreover, when the size of the object is adequate and/or the shape thereof matches the profile, the object can be further secured/fitted between the auricle and the skull so as to achieve a greater securement effect. Consequently, there are many selections.

Here, the electrode mounted on the extension member also can be selected to adopt the above-mentioned contact assurance structure. For example, the electrodes can be configured as scattered electrodes, and/or shrinkable electrode for adapting the shape of the auricle backside and/or the V-shaped recess, thereby facilitating to maintain the contact between the electrode and the skin.

In addition to the aforementioned in-ear housing and hook-typed structure, there still have other possibilities for securing electrode.

For example, magnetic attraction method can be used to achieve the securement effect. One possibility is the front ear member and the extension member are configured to magnetically attract to each other across the auricle, which also can achieve the securement effect. In such embodiment, the two members can be configured to be of magnetism, or they can be made of magnetic attractable material. For example, one member can be configured to have magnetism, and the other member can be attracted magnetically; alternatively, the two members can be configured to have magnetism. There is no limitation. Furthermore, preferably, a portion of the extension member can be made of a flexible material, such as a connecting wire, for improving the comfortableness during usage. Particularly, since magnetism is used to achieve the securement, in addition that the extension member can extend upward, cross over the top of the auricle and reach the backside of auricle, it can also be configured to extend downward, cross the bottom of the auricle, and reach the backside of auricle. Therefore, greater implementation possibility is provided.

Furthermore, alternatively, a clamp can be used to achieve the aforementioned electrode installations as using the magnetism. With the clamping force generated by the clamp, the effect of maintaining the location of electrode and stabilizing the electrode contact can be achieved at the same time. Therefore, there is no limitation.

For such method (with the use of magnetism and/or clamping force for securement), it is advantageous that only one single dimension is sufficient for adapting to different sizes of auricles such that the manufacturing thereof becomes more convenient, and also, changing the electrode installation location becomes possible, which maximize the usage value thereof.

Furthermore, particularly, the electrode contact location of the present invention can also be achieved via an eyeglass structure. Generally, while wearing the eyeglasses, the locations naturally contacted thereby include, but not limited to, the nasal bridge, the nasion and/or the region between two eyes contacted by the nose pad, the area adjacent to the temple contacted by the front section of the eyeglass temple, the V-shaped recess area between the auricle and skull contacted by the rear section of the eyeglass temple, and the auricle backside contacted by the end portion of the eyeglass temple that is located behind the auricle. Among these locations, there are electrode contact locations matching the ones claimed by the present invention. Accordingly, the electrode of the present invention can be naturally configured to mount on the eyeglass structure, and through the action of wearing the eyeglass structure, the electrode contact can be achieved. Therefore, it is also a convenient choice to users. In addition, since the supporting locations of the eyeglass structure and the head include at least three locations including two auricles and nose, it can be stably arranged on the head without movements, so that the forces can be applied naturally to maintain the stable contact between the electrode and the skin, which is a relatively advantageous embodiment.

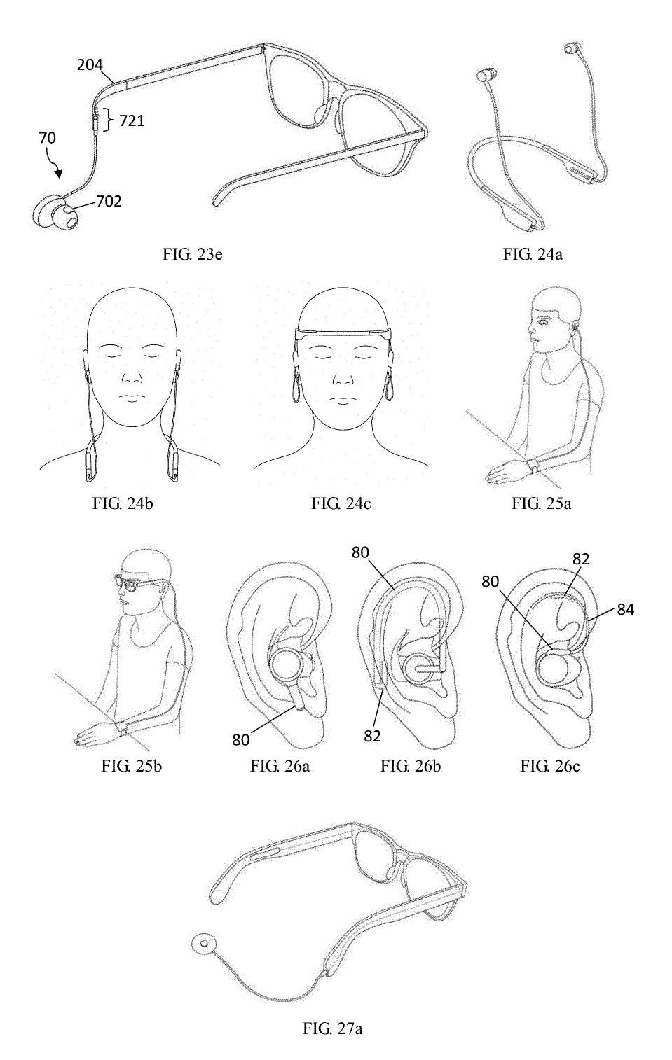

The eyeglass structure described here refers to a wearing structure that uses the auricles and nose as the supporting points for wearing onto the head such that it is able to contact with the skins of the head and/or ears. Accordingly, the present invention is not limited to the conventional eyeglass structure, and variations thereof are also possible. For example, it can be a structure with clamping force toward the two sides of the skull, or it can be an elastic continuous members without temple hinges, as shown in FIG. 23d; or it can have an temple structure extended to the occipital lobe at the rear of the head, or it can be configured to have unsymmetrical temples, such as one temple with bending portion and the other temple without bending but placed on top of the auricle only; or it can be arranged with straps for connecting two temples in order to enhance the securement effect. In addition, the eyeglass structure may not contain glasses. Furthermore, the nose pad is also not limited to any specific types, and as long as it contacts the nasal bridge, the nasion and/or the region between two eyes, it can be regarded as part of the nose pad. Moreover, the contact location of the eyeglass structure with the head/ears is not limited to any specific location, for example, some eyeglasses may be configured to contact other locations surrounding the eyes due to the actual needs of use or style, such as VR glasses. Consequently, there are various different possible embodiments, without limitation.

In terms of the selection of material, in addition to the rigid materials of conventional eyeglasses, the material can also be a flexible material such that the stability of electrode contact can be enhanced and the usage comfortableness can also be provided. For example, memory metal or flexible plastic material can be used to form the glass frame, and/or the electrode contact location can be arranged with elastic rubber or silicon in order to provide greater contact stability. Therefore, there is no limitation.

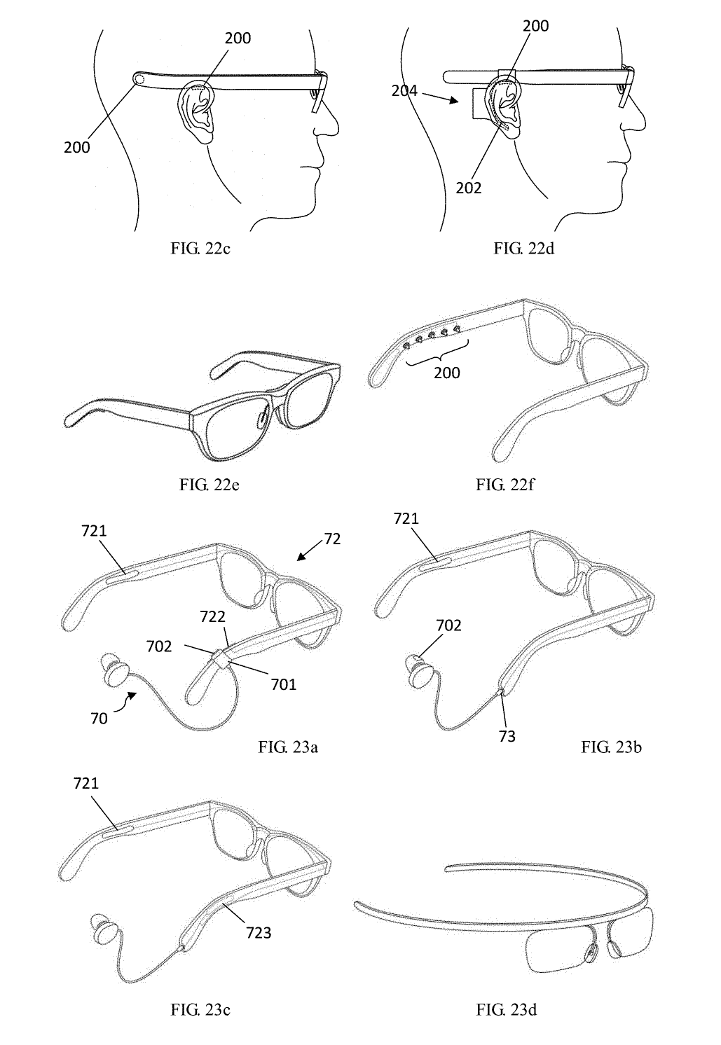

As for the mounting methods of the electrode onto the eyeglass structure and the required circuits (such as, processor, battery, wireless transmission module), there are also various possibilities. For example, one of the methods is that, as shown in FIGS. 22a, 22c and 22e, the required circuits are directly embedded in the eyeglass structure, and the electrode is directly exposed at the surface of the temple and/or the glass frame so as to contact with the skin of the skull and/or ears during the wearing thereof.

Another feasible method is to achieve the configuration of the electrode and circuits through attachment structure. In one embodiment, preferably, the engagement structure can be configured to receive at least a portion of the circuits thereby simplifying the manufacturing complexity of the eyeglass structure. For example, as shown in FIG. 22b, the engagement structure is configured to electrically connect to the eyeglass structure so as to allow the electrode 202 thereon and the electrode 200 on the eyeglass structure to perform signal acquisition. Alternatively, as shown in FIG. 22d, the engagement structure can also be configured to include two electrodes 200, 202, and through the engagement structure is engaged with the eyeglass stricture, the two electrodes can be installed on the auricle. In the above-described two methods, physiological signals are both acquired through contacting electrodes with one single side of the auricle. In addition, the number of the engagement structure can also be plural, for example, both temples can be attached with one attachment structure respectively, and thus, signal acquisition can be performed by the electrodes respectively thereon contacting with the two auricles and/or the nearby skull. Under such condition, the electrical connection between the two attachment structures can be achieved via the eyeglass structure, or via a connecting wire connected therebetween, and the required circuits can be partially or completely mounted in the eyeglass structure or the attachment structure depending upon the needs. Furthermore, the attaching location of the engagement structure is also not limited to locate behind the ear. For example, it can also be attached at the side portion of the head that is in front of the ear, or attached at the front side and behind the ear at the same time as long as the usage is not affected, without limitation. Moreover, the engagement structure can also be used only for mounting the circuits in such a way that after being electrically connected with the eyeglass structure, the electrodes on the eyeglass can be driven to perform signal acquisition. In addition, the engagement structure can be configured to be removable, so that the user can select to attach the engagement structure onto the eyeglass structure as there is a need to perform the measurement.

Furthermore, another feasible method is to combine the eyeglass structure and the ear-worn structure for mounting the electrodes and circuits. The advantage of using the ear-worn structure is that the ear-worn structure is already equipped with the structure for stably positioning on the ear such that it is convenient to use. In addition, since the distance between the ear-worn structure and the eyeglass structure is short, if a connecting wire is used for connecting therebetween, it is still appropriate. Furthermore, the cooperation between the eyeglass structure and the ear-worn structure can also provide a greater range for the installation of electrodes such that the types of signals capable of being acquired are also increased. Therefore, it is advantageous. In practice, the ear-worn structure can be made in accordance with the aforementioned embodiments of the attachment structure, for example, it can be arranged on one single side or two sides of the head, and the surface thereof can be provided with or without electrodes, and/or it can be configured to be removable or not. Consequently, there are various possibilities without limitation.

When the eyeglass structure is used for mounting electrodes, for the electrical connection between the electrodes and circuits, in addition to the use of wiring embedded in the eyeglass structure, the original conductive part of the eyeglass structure can also be used to achieve such connection. For example, eyeglasses made of conductive material, such as metal eyeglasses, can be used, and the existing conductive part in the eyeglass structure can also be used, such as, the metal hinge structures for connecting the front glass frame and the two temples, the existing metal conductive parts in the glass frame, the metal nose pad and/or the existing metal conductive part in the temples are all. Through such configuration, even a conventional eyeglass structure can be used to acquire the physiological signals, so that advantageously, it can be widely accepted by the public owing to its general appearance.

In addition, for the electrodes configured to mount on the eyeglass structure, the aforementioned contact assurance structure is also applicable, for example, the scattered electrodes, electrode protrusions, and/or shrinkable electrodes. Thus, in addition to being able to adapt to the shape of the auricle backside and/or the V-shaped recess area, particularly, when hairs occur at the electrode contact locations, the scattered, protruded and shrinkable structures all will facilitate the electrodes to penetrate through the hairs such that the contact difficulty can be reduced. As shown in FIG. 22f, the temple is disposed of a plurality of scattered and shrinkable electrodes. Besides, with forming the electrode into plural scattered contact points, the contact range of the electrode can be expanded, so that it is able to overcome the size difference between different users' heads, which is an advantageous.

It shall be noted despite that specific embodiments of the present invention are described, it can be understood that these embodiments are provided as examples only, and the present invention shall not be limited to such embodiments. As long as an eyeglass structure or ear-worn structure which is supported by the ear can achieve the contact between the electrode and the skin covering the ear cartilage, such structure shall be within the scope of the present invention. In addition, different embodiments can also be combined without limitation.

Moreover, since the purpose of the present invention is to allow users to obtain EEG signals via wearable device at any time, it is preferable to use dry electrodes, such as conductive metal, conductive rubber, conductive silicon, conductive foam and conductive fiber, thereby maximizing the convenience.

In the following, the possible configurations of electrode arrangements for detecting brain activities are described.

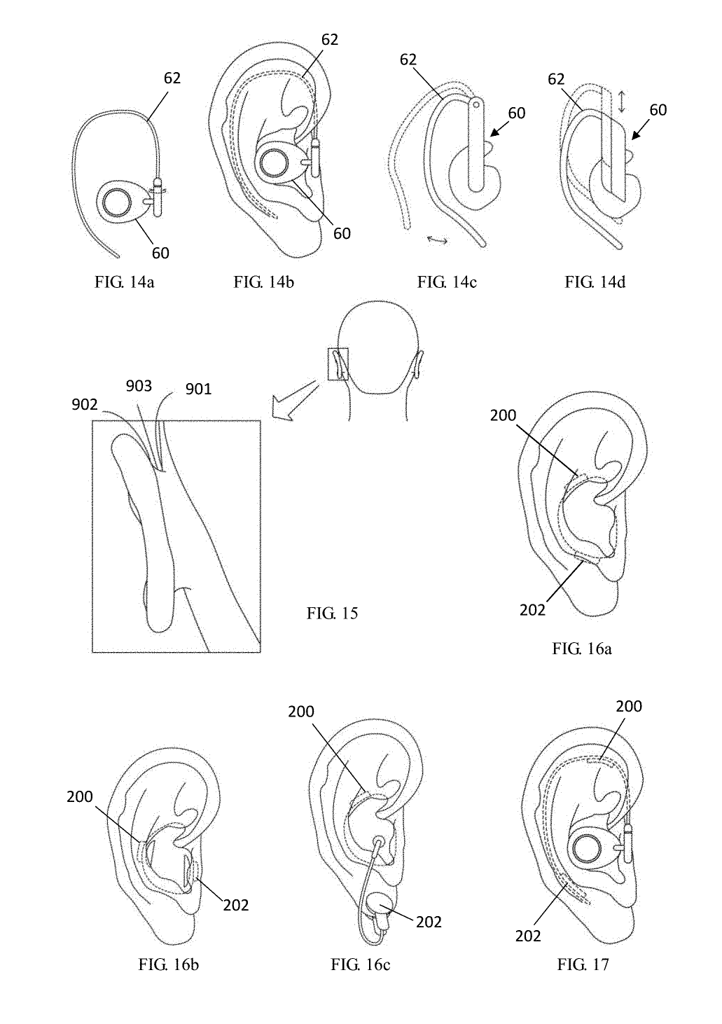

Please refer to FIGS. 16a-16b, showing illustrations of two electrodes configured on an in-ear housing at the same time. As mentioned earlier, the upper portion of the auricle and the lower portion of the auricle can be used as the locations for the installation of activity detection electrode 200 and the reference electrode 202. Therefore, as long as the contact positions of the in-ear housing with the auricle inner side are appropriate, single in-ear housing can also complete the installation of two electrodes necessary for obtaining EEG signals.

As mentioned above, for acquiring EEG signals through two electrodes, except that the distance between two electrodes should be sufficient, if each electrode can have sufficient independence, it can also be an effective method for obtaining EEG signals. Accordingly, even though the contacting range of one single in-ear housing is small, since the physiological structure of the ear canal creates a space separation, it is still sufficient to acquire EEG signals for analysis.

Therefore, the two electrodes in FIG. 16a are respectively located at the upper portion and the lower portion of the in-ear housing in order to contact the concha wall on the top and the antitragus/intertragic notch at the bottom, wherein the upper electrode can be used as the activity detection electrode, and the lower electrode can be used as the reference electrode. Furthermore, in FIG. 16b, one electrode contacts the tragus for being the reference electrode, and the other electrode contacts the concha wall opposite from the location of the tragus for being the activity detection electrode. Alternatively, the reference electrode which contacts the tragus, the intertragic notch and/or the antitragus can cooperate with the activity detection electrode which is arranged to contact the concha floor, for example, the in-ear housing as shown in FIG. 6b can be used to obtain EEG signals under this condition. When determining the locations of the activity detection electrode and the reference electrode, it is preferable to distribute two electrodes at two opposite sides of the ear canal so as to facilitate the acquisition of effective EEG signals.

In this embodiment, the in-ear housing can be configured to only carry the electrodes and connect to a host machine having the required circuits, such as, processor, battery and wireless transmission module, accommodated therein. As to the position of the host machine, there is no limitation, for example, it can be placed at the rear of ear, or can be worn by the body, e.g., through being embodied as neck-worn type, eyeglass type, head-mount type, wrist-worn type, or arm-worn type. Alternatively, the in-ear housing can also be configured to directly accommodate the required circuits therein. Therefore, the present invention can be modified depending upon the actual needs without limitation.

In addition, the in-ear housing can also be configured to carry one single electrode only for contacting the concha wall, the antitragus, the tragus, and/or the intertragic notch. For example, the electrode on the in-ear housing can cooperate with an electrode directly arranged on the skull, such as an electrode which is installed at the parietal lobe, the frontal lobe and/or the occipital lobe through a wearable structure, e.g., headband, headgear and patch etc., so as to detect the brain activities. In this embodiment, preferably, the electrode on the in-ear housing is implemented to be the reference electrode. Alternatively, the electrode on the in-ear housing can also be implemented to be the activity detection electrode for cooperating with the reference electrode arranged inside the ear clamp on the ear lobe (as shown in FIG. 16c). Certainly, it also can be configured to employ two in-ear housings respectively on two ears for carrying one electrode each, for example, it can be implemented that the electrode on one in-ear housing is used as the reference electrode (i.e. contacting the lower portion of the auricle), and the other electrode on the other in-ear housing is used as the activity detention electrode (i.e. contacting the upper portion of the auricle). Nevertheless, it shall be noted that since there is sufficient distance between the two ears, the contact positions of the electrodes are not limited. Consequently, no matter the electrodes on two in-ear housings contact the upper portion or the lower portion of the auricles, it is still able to acquire EEG signals for analysis, for example, the electrode on one of the in-ear housings can contact with the skin at the upper portion of one auricle and the other electrode on the other in-ear housing can contact with the skin of the lower portion of the other auricle, or both can contact with the skin of the upper portions of the two auricles. Alternatively, the electrode can also be configured to contact with the concha floor (as shown in FIG. 6b), for example, the electrode on one of the in-hear housings can contact with the concha wall, the antitragus, the intertragic notch and/or the tragus, whereas the electrode on the other in-ear housing can contact with the concha floor, or both contact the concha floors. Therefore, there are various possibilities without limitation.

Next, when the hook-typed structure is employed, the electrode on the extension member can selectively contact with the locations of the V-shaped recess, the upper portion of the auricle backside and/or the lower portion of the auricle backside depending upon the needs. As shown in FIG. 17, the two electrodes are mounted on the extension members, wherein one electrode contacts the skin of the V-shaped recess between the auricle and the skull and/or the upper portion of the auricle backside for being used as the activity detection electrode 200, and the other electrode contacts the skin of the lower portion of the auricle backside for being used as the reference electrode 202. Alternatively, the electrode on the extension member can cooperate with the electrode directly arranged on the skull, such as, an electrode which is installed at the parietal lobe, the frontal lobe and/or the occipital lobe through a wearable structure, e.g., headband, headgear and patch etc., so as to detect the brain activities. Furthermore, in this embodiment, preferably, the electrode on the extension member is implemented as the reference electrode. Alternatively, the electrode on the extension member can also cooperate with the reference electrode arranged on the earlobe through the ear clamp so as to acquire EEG signals. Furthermore, it also can be implemented to employ two ear hooks and the extension member of each ear hook has one electrode mounted thereon, for example, it can be configured that one electrode is used as the reference electrode (i.e. contacting the lower portion of the auricle backside), and the other electrode is used as the activity detection electrode (i.e. contacting the V-shaped recess and/or the upper portion of the auricle backside). And, similarly, since there is sufficient distance between two ears, the contact locations of the electrodes are not limited, and no matter the contact locations of the electrodes on two extension members are the upper portions or lower portions of the auricles, sufficient EEG signal can be acquired for analysis without limitations.

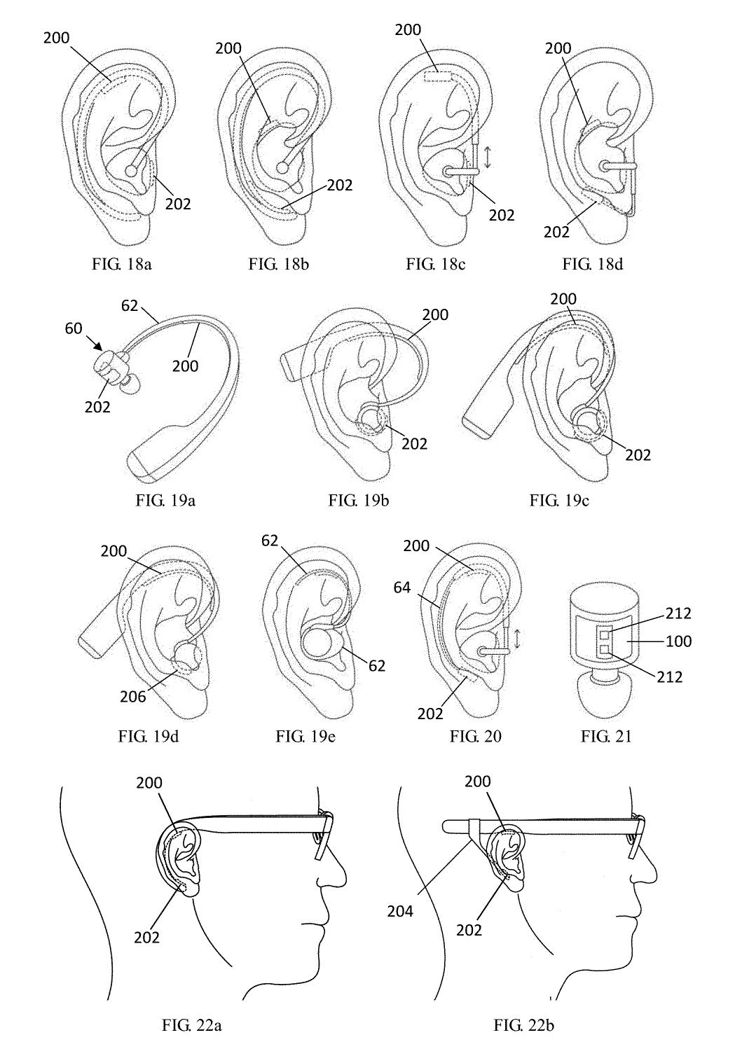

Furthermore, FIGS. 18a-18d illustrate other possible embodiments of the present invention. FIG. 18a illustrates the embodiment where the in-ear housing contacts with the tragus or the intertragic notch at the lower portion of the auricle inner side, and the extension member contacts with the V-shaped recess and/or the upper portion of the auricle backside. In this embodiment, the electrode on the extension member not only contacts with the skin of the V-shaped recess and/or the auricle backside, it can also be configured to contact the skin of the skull without limitation. Furthermore, FIG. 18b illustrates the embodiment where the in-ear housing contacts the concha wall of the upper portion of the auricle inner side, and the extension member contacts the lower portion of the auricle backside. Moreover, it can also be configured as the electrode on the in-ear housing contacts the concha floor (e.g., by utilizing the in-ear housing as shown in FIG. 6b), and the electrode on the extension member contacts with the V-shaped recess or skull, or the auricle backside. Alternatively, it can also be configured in such a way that an ear clamp is extended from the extension member to arrange the electrode on the earlobe, so as to cooperate with another electrode on the in-ear housing contacting with the upper portion of concha wall and/or the concha floor of the auricle inner side for acquiring EEG signals. It shall be noted that, the electrodes at the inner side and the backside of the auricle are preferably distributed at two opposite sides of the ear canal, so as to ensure the space separation necessary for signal acquisition.

In another preferred embodiment, as shown in FIG. 18c, the length of the extension member can be shortened, and an adjustment mechanism can be employed to allow the extension member to move vertically. Therefore, the electrode contacts can be more stable and can adapt to various auricle dimensions of different users. In this example, the electrode on the in-ear housing is configured to be the reference electrode 202 to contact the location of the tragus and/or the intertragic notch; whereas the electrode contacting the V-shaped recess and/or the auricle backside is configured to be the activity detection electrode 200 such that it can contact the skin of the V-shaped recess and/or auricle backside or the skin of the skull without limitation. Furthermore, in another preferred embodiment, as shown in FIG. 18d, the extension member is configured to locate at the lower portion of the in-ear housing, so as to allow the electrode thereon to contact the lower portion of the auricle, such as the auricle backside skin above the earlobe. In addition, similarly, the adjustment mechanism can also be employed to achieve the vertical movement, so as to increase the contact stability and to adapt to different auricle dimensions.

Alternatively, another embodiment as shown in FIG. 19a is also possible. In this embodiment, a portion of the front ear member 60 not entering into the ear canal can be configured to have a smooth curve, such as a cylinder, and the extension member 62 can also be configured to have a smooth curve. The electrodes 202, 200 are mounted respectively on the surface of the portion not entering into the ear canal and on the surface of the extension member facing toward the V-shaped recess/auricle backside. Under such condition, as long as the distribution range of the electrodes is enough, it is able to adapt to different auricle dimensions of different users by easily rotating the entire ear-worn structure, such as rotating with the cylinder as the center. For example, FIG. 19b illustrates the condition where the device is arranged on a relatively larger auricle, and FIG. 19c illustrates the condition where the device is arranged on a relatively smaller auricle. As shown, it can be understood that, since the distribution range of the electrodes 200, 202 is enough to cover the movement generated due to the rotation, while adapting to different dimensions of auricles, such design also can ensure the contact between the electrode and the skin. Further, for simplifying the manufacturing thereof, the electrodes can be configured to cover the entire outer surface of the cylinder and/or the entire surface of the extension member facing toward the V-shaped recess, for example, they can be made of conductive material. Consequently, there are various possibilities without limitation.

Furthermore, in this example, when an angle is formed between the portion entering into the ear-canal and the portion not entering into the ear canal, the action of placing into the ear canal can naturally allow the portion not entering to the ear canal to be firmly maintained on the auricle inner side, and also can apply a force toward the direction of the tragus which further facilitates the electrode contact stability. Furthermore, different dimensions of the portion entering into the ear canal can be provided for different users, which can also facilitate the portion not entering into the ear canal to be firmly maintained at the auricle inner side.

Moreover, the electrode on the portion not entering into the ear canal can also be configured to contact the tragus. For example, through adjusting the angle of the in-ear housing, the portion not entering into the ear-canal can be directed to face toward the antitragus. Under such condition, as long as the portion entering into the ear canal is made of an elastic material, it would not cause any pressure on the ear canal, and the portion not entering the ear canal can be naturally locked into the space between the antitragus and the ear canal, so as to achieve a stable installation. Furthermore, to increase the contact stability between the electrode and the antitragus, it can also employ an additional protrusion to further ensure the contact, as shown in FIG. 19d, the protrusion 206 for mounting the electrode can be made of an elastic material. Therefore, there is no limitation.

Moreover, the extension member can also be configured to apply a force toward the V-shaped recess/auricle backside so as to ensure the contact between the electrode thereon and the skin, for example, it can be made of an elastic material, such as elastic metal, elastic rubber etc. As shown in FIG. 19e, the extension member is configured to be of restoring force such that after it is pulled for placing onto the auricle, it is able to restore back to its original shape and closely attach onto the auricle backside, so as to achieve the stable contact between the electrode and the skin.

When the aforementioned extension member is only provided with the function of electrode, namely, most of the circuits are located inside the in-ear housing, the extension member can be further configured to be removable, such as through setting a connection port. Consequently, it can achieve the merits of convenient storage and portability. In practice, for example, the extension member can be configured to be made of an elastic conductive material, such as the elastic steel, memory metal, conductive rubber and conductive silicon, for being used as the electrode directly; or the extension member can also be configured to complete the electrical connection between the electrode thereon and the circuits inside the in-ear housing after finishing the connection thereof with the in-ear housing.

In addition, through such removable configuration, another embodiment of the present invention is also possible, namely, the electrode on the extension member can be used as an extension of the electrode on the in-ear housing. For example, when the in-ear housing includes two electrodes, then the external connection of the extension member can be used to replace one of the electrodes such that it can be used as another choice for contacting. For example, the contact with the auricle inner side is changed to the contact with the V-shaped recess/auricle backside. In addition, it also can provide another choice for securement, for example, the force from the extension member to apply on the in-ear housing can be increased. Alternatively, the extension member can also be used as an extension securement structure only in order to further increase the securement force between the in-ear housing and the auricle. Therefore, there are different possibilities depending upon the needs without limitations.

In still another preferred embodiment, as shown in FIG. 20, the in-ear housing does not have electrode arranged thereon but is used for securement, and also provides a magnetic force for attracting the electrode in contact with the lower portion of the auricle backside; whereas the other electrode is carried by the extension member extending out of the in-ear housing to contact with the V-shaped recess and/or the upper portion of the auricle backside. Particularly, an adjustment mechanism can be employed between the extension member and the in-ear housing for adapting to different ear sizes, and the electrode contacting the lower portion of the auricle can use a connecting wire 64 or a flexible material to connect to the extension member. As a result, despite that the extension member may be moved due to the adjustment mechanism, the contact location of the lower electrode would not be affected. Consequently, not only the contact stability of two electrodes can be ensured, but also the effect of adapting to different auricle dimensions can be achieved, which is of great advantage. Alternatively, preferably, the extension member extending from the in-ear housing can also be configured to bend along with the shape of the auricle. For example, it can be directly configured as a connecting wire or can be made of elastic material; therefore, when the electrode contacting with the lower portion of the auricle backside is secured through the magnetic force, the electrode contacting the V-shaped recess and/or the upper portion of the auricle backside not only can be properly arranged between the auricle and the skull, but also can be further stabilized by the pulling force generated due to the magnetic attraction. Furthermore, the extension member can also be configured to be replaceable, such as for different lengths or different materials, so as to adapt to different users.

It shall be noted that for any one of the embodiments mentioned above, the material and shape of the extension member can be modified depending upon different implementation conditions. For example, the extension member can be made of an elastic material equipped with restoring force, such as elastic metal, elastic plastic, silicon etc., in order to ensure that the electrode is always maintained by the contact force toward the auricle backside. Alternatively, the extension member can be made of a material with plasticity, such as memory metal, plastic with pliability, so as to be bent by the user depending upon the shape of the auricle and also ensure the contact stability. Therefore, there are various possible embodiments without limitation.

Furthermore, the circuits required for acquiring physiological signals, such as processor, battery and wireless transmission module, can be accommodated inside the front ear member or in a housing at the rear of the ear, or in a host machine which can be worn on the user and is connected through a connecting wire, such as wrist-worn type, neck-worn type, head-mount type, eyeglass type or arm-worn type, without limitation.

In a preferred embodiment, particularly, no matter the configuration of only employing the in-ear housing or the configuration of having the extension member, the host machine both can be further implemented to be a wearable structure suitable for the neck and the head, as shown in FIGS. 24a-24c. Namely, the wearable structure can be selectively arranged at the neck or the head depending upon the user's needs. In addition, when being worn on the head, it can be further selected to arrange the wearable structure at the forehead (FIG. 24c), on the top of the head or at the rear of the head, without limitation.

Here, the wearable structure is configured to have two end portions and a bending portion connecting the two end portions, namely, it has a shape similar to C. Through such bending portion, the wearable structure can be adaptively arranged at the neck or the head. Therefore, preferably, the bending portion can at least partially match with the curve at the rear of the neck, in such a way that when the wearable structure surrounds the neck, the two end portions can be positioned at the two sides and/or the front thereof to form a stable installation. Furthermore, when it is installed on the head, the bending portion can match with the front, the top and/or the rear curve of the head while the two end portions fall at two sides of the head, thereby achieving a stable engagement with the head.

First, when implementing to be the neck-worn type, since it uses the neck as the support, the size and shape of the host machine can have greater variations. Furthermore, in comparison to the arrangement on the arm or wrist, not only the length of the connecting wire with the ear-waring structure is shortened, but also the activities of the hand is not affected by the connecting wire, which provide more convenience. Moreover, in comparison to where the host machine is implemented as the in-ear housing or to be located at the rear of the ear, this configuration is able to reduce the burden on the ear and is also able to increase the installation stability due to the reduction of the size of the in-ear housing. Further, such kind of neck-worn type device is similar to the conventional necklace, and users can be adapted to such wearing easily.

Furthermore, when implementing to be the head-mount type, because the locations thereof contacting with the head is increased, the possibility of obtaining more EEG signals from different cortices also increases. Therefore, through selecting different wearing locations, the user can determine the acquisition of different kinds of EEG signals. For example, referring to FIG. 1, when the electrode is arranged at the forehead, it is able to acquire EEG signals of the frontal lobe; when it is arranged at on top of the head, it is able to acquire EEG signals of the parietal lobe; when it is arranged at the rear of the head, it is able to acquire EEG signals of the occipital lobe; when the electrode is arranged on the two end portions, it is able to acquire EEG signals of the temporal lobe. In addition, when the electrode is arranged on the location in contact with the surrounding of the eyes, such as the forehead, the temples etc., it is also able to acquire EOG (Electrooculography) signals at the same time.

Moreover, the electrode contacting with the head can also be configured to acquire EEG signals by cooperating with the electrode on the ear-worn structure without limitation. And, when the electrode contact locations of the wearable structure contains hairs, such as the top of the head, the rear of the head, two sides of the head, then as mentioned above, the contact assurance structure can be used, such as the scattered electrodes, protruded electrodes and/or shrinkable electrodes, in order to facilitate the electrodes to penetrate through the hairs and to reduce the contact difficulty between the electrode and the skin.

As to how the wearable structure be adaptively worn on the neck and the head, there are many possibilities. For example, it can be achieved through the selection of materials, such as, a material with elasticity can be selected to apply force on two sides of the head, so as to achieve the securement effect, e.g., elastic steel and elastic plastic. It can also be achieved through structural design, for example, it can be designed to arrange on the auricle or to equip with an anti-movement structure. Further, it can utilize an assisting member to achieve the stable contact with the head, for example, a structure capable of tightening the two end portions, such as an elastic strap, or a buffering structure mounted at the inner side of the wearable structure can be utilized to help the wearable structure to be stably maintained on the head. Thus, there is no limitation. Furthermore, if the circuits are mainly distributed at the two end portions, then it can be further configured that the bending portion is replaceable, so as to enable the changes of different shapes, materials, dimensions and colors, which provides a more convenient usage. In addition, oppositely, the two end portions can also be implemented as replaceable, such that through the changes of different circuits, the executable functions thereof can be changed. Therefore, there are various possibilities without limitation.

Consequently, with such structural design, since it is similar to wear a conventional necklace, users would not feel any additional burden. Further, the space for accommodating the circuits is also increased, such that more functions can be provided, for example, battery with large capacity can be provided to increase the usage time, music playing function can be provided, GPS positioning function can be provided, and/or a control interface as shown in FIG. 24a can be provided at the two end portions where can be easily contacted by the user. All are choices with great advantages.

In addition, it is particularly advantageous when being configured as the wrist-worn type. Since the wrist-worn device, such as, wristband and watch, is one of most common used portable information providing interface for users, through arranging the host machine on the wrist along with the additional information providing interface, users are able to obtain information whenever necessary, which is similar to using the watch. Therefore, the condition of use will be like FIG. 25a. Normally, users can wear the watch/wristband equipped with EEG signals acquiring function on the wrist. When there is a need to measure EEG signals, the user only needs to further connect the EEG electrodes and arrange them on the ear, and then, it becomes a wrist-worn EEG monitoring device for portable use. In this embodiment, the electrodes connected can be any one of the ear-worn types mentioned above, such as it can be electrodes that are provided on one single ear-worn structure, or two ear-worn structures each equipped with one electrode, which depends upon the actual needs. If the design of a single ear-worn structure comprising two EEG electrodes is used, then only one connecting wire is required; therefore, the convenience is further increased, and the complexity is also significantly reduced. Furthermore, when two ear-worn structures are used, it can be further configured to obtain two-channel EEG signals, which is able to be used to monitor the activities of left brain and right brain. Accordingly, no matter which type of electrodes is used, both are advantageous.

When the electrodes are configured to mount on the eyeglass type, similarly there are various choices. For example, as shown in FIG. 22a, the activity detection electrode 200 can be installed on the temple to contact the V-shaped recess and/or the upper portion of the auricle backside (upper portion of auricle), and the reference electrode 202 can be arranged at the rear bending portion of the temple to contact the lower portion of the auricle backside (the lower portion of the auricle). Furthermore, the rear bending portion of the temple can be configured to have elasticity for increasing the contact stability of the electrode. Alternatively, as shown in FIG. 22b, one electrode can be mounted one side of the temple to contact the V-shap recess and/or the upper portion of the auricle backside, and another electrode can be mounted on an engagement structure 204 which is engaged with the same temple so as to contact the auricle backside. Here, the engagement structure can contact any portion of the auricle backside, or it can also be configured to engage with the other temple of the eyeglass without limitation. Alternatively, as shown in FIG. 22c, one electrode can be mounted at the rear end of a temple which extends to the back of the head, for contacting the skull corresponding to the occipital lobe, and another electrode can be mounted on the same temple or the other temple for contacting the V-shaped recess and/or the upper portion of the auricle backside. Such configuration is particularly suitable for the eyeglass structure without hinges, as shown in FIG. 23d, whose original temples already extend to the rear of the head. Alternatively, as shown in FIG. 22d, the electrodes on the engagement structure 204 engaged with the temple can be implemented to contact the V-shaped recess and/or the upper portion of the auricle backside as well as to contact the lower portion of the auricle backside. Alternatively, the two electrodes can be respectively arranged on the two temples for contacting the V-shaped recesses and/or the upper portions of the auricle backside at two sides. Alternatively, the rear end of the temple also can be implemented to bend, so that one single temple can have two electrodes to contact the V-shaped recesses and/or the upper portions of the auricle backside and the lower portion of the auricle backside (as shown in FIG. 22a). Alternatively, since there is sufficient distance between the two ears, the both temples can also be configured to bend for contacting the lower portion of the auricle backside. Alternatively, the engagement structure can be engaged on one single temple or two temples to contact the lower portion of the auricle backside (as shown in FIG. 22b), without limitation. Alternatively, as shown in FIG. 22e, the electrodes can also be mounted at a location for contacting the nasal bridge/nasion/area between two eyes, as well as a location for contacting the V-shaped recess and/or the upper portion of the auricle backside or the lower portion of the auricle backside, so as to perform the brain activity detection. Accordingly, there are various possibilities without limitations, and as long as the contacts between electrodes and the skull and/or the auricle that can be achieved by the eyeglass structure shall be within the scope of the present invention. In addition, the aforementioned locations and configurations of the electrodes are provided for the purpose of illustration only, which can be substituted and/or combined with each other without limitations.

Particularly, when the electrode is located at the surrounding of eyes, as shown in FIG. 22e, such as the nasal bridge/nasion/area between two eyes, temples, then the Electrooculography (EOG) signals can also be acquired; wherein the EOG is to measure the corneo-retinal standing potential that exists between the front and the back of the human eye, which can be used to measure the location of the eyeball and the physiological change of eye movements. Because EOG signals and EEG signals are of different frequencies and amplitudes, they can be separated from each other via signal processing. Therefore, under the concept of the present invention, to acquire these two types of signals, only a minimum number of two electrodes are required. For example, it only need to arrange one of the electrodes at the location for contacting the nasal bridge/nasion/area between two eyes or the temple, along with arranging another electrode at the location for contacting the auricle inner side, the auricle backside and/or the V-shaped recess, then simultaneously, EEG signals and EOG signals can be acquired without other special arrangements. In addition, such configuration is particularly suitable for being used in the eyeglass structure, so that users only need to wear the eyeglasses and the measurements of two kinds of signals can be performed without redundant steps, which is convenient to the users.

Moreover, in a special embodiment, it can be configured to have a plurality of electrodes arranged at two sides of the eyeglasses so as to acquire signals of the left brain and the right brain respectively. For example, two electrodes can be disposed on the temple and/or the glass frame at the right, and the other two electrodes can be disposed on the temple and/or glass frame on the left side. As a result, as long as the circuits are separated, the eyeglass structure becomes a two-channel EEG signal acquisition device, which is advantageous. Under such condition, the layout of the circuits can be directly arranged on the left and right portions of the eyeglass structure, or alternatively, external module(s) comprising the circuits can be used to connect with the temple(s). There is no limitation.

Furthermore, the installations of two electrodes used for acquiring EEG signals can also be achieved by the eyeglass structure and the ear-worn structure, for example, an ear-worn structure can be extended from the eyeglass structure, or the eyeglass structure can be equipped with a connection port for electrically connecting with an ear-worn structure. Therefore, through the use of eyeglass structure, it is able to selectively contact with the V-shaped recess, the auricle backside, the temple, the nasal bridge, the nasion and/or the area between two eyes, and through the use of ear-worn structure, it is able to selectively contact the V-shaped recess, the auricle backside, the concha floor, the concha wall, the antitragus, the intertragic notch, and/or the tragus, thereby jointly achieving EEG signal acquisition. Here, the ear-worn structure can be the in-ear housing or the ear hook, without limitation.

In the present invention, the eyeglass structure, the ear-worn structure and the electrodes can also have different configuration selections. For example, in a preferred embodiment, as shown in FIG. 23a, one electrode is located on a temple of the eyeglass structure, the other electrode is located on the ear-worn structure and the circuits are arranged inside the ear-worn structure, wherein the electrode 721 is mounted at a contact position of the eyeglass structure 72 where provides the securement force for contacting the head and/or the auricleas as being worn on the head, and the other electrode 702 is mounted on a surface of an engagement structure 701 of the ear-worn structure 70 engaging with a temple of the ear-worn structure 70, so as to contact the skull and/or the auricle as the ear-worn structure and the eyeglass structure are combined together. Under such condition, for connecting with the ear-worn structure, the eyeglass structure can be equipped with an electrical contact area 722 at the temple for engaging the ear-worn structure. The electrical contact area 722, in addition to electrically connecting with the circuits inside the ear-worn structure and the electrode 702 on the surface thereof, also electrically connects to the electrode 721 at the other temple, thereby achieving the sampling loop. Alternatively, the electrode 721 can also be arranged on the glass frame so as to form a sampling loop with the electrode 702 on the ear-worn structure. Consequently, the configuration thereof can be modified according to the actual needs without any limitations.

Moreover, the engagement between the ear-worn structure and the eyeglass structure can have different choices. For example, as shown in FIG. 23b, the rear end of the eyeglass temple can be configured to have a connection port 73, thereby achieving a mechanical connection and an electrical connection simultaneously with the ear-worn structure via a plug in connection. In this embodiment, the electrode 702 of the ear-worn structure is arranged on the surface of the in-ear housing of the ear-worn structure.

Furthermore, it can also be configured to have two electrodes arranged on the surface of the eyeglass structure. As shown in FIG. 23c, the two temples can be equipped with the electrodes 721, 723 thereon respectively; or the two electrodes can be arranged on one temple and on the glass frame respectively. Accordingly, by connecting to the ear-worn structure, the connection with the circuits inside the ear-worn structure can be completed and the electrical physiological signal acquisition can be performed. In addition, the electrode can also be arranged on the ear-worn structure, and as a result, the electrode on the ear-worn structure can be regarded as the reference electrode, and the electrodes 721, 723 can be used as the activity detection electrodes, so as to respectively or simultaneously acquire EEG signals of the temporal lobes at two sides.

It shall be noted that although the embodiments shown in FIGS. 23a and 23c are of the configurations where two electrodes are disposed on two temples, the present invention shall not be limited thereto. The two electrodes can also be configured to dispose on one temple and on the glass frame. Furthermore, it can also be configured to have more than two electrodes, for example, the two temples and the glass frame all can have electrode(s) disposed thereon. There is no limitation. Moreover, the combination of the ear-worn structure and the eyeglass structure can also have various possibilities. In addition to employing the connection port or slipping-on as shown, there are also other choices, such as magnetic attraction, locking or sliding slot, without limitation. Furthermore, for the eyeglass structure, in addition to the traditional eyeglasses type as shown, the aforementioned eyeglass structure without hinges can also be used, such as the elastic continuous member without hinges shown in FIG. 23d, and/or the eyeglass structure without lens. Therefore, the present invention can be modified according to the actual needs.

In another preferred embodiment, as shown in FIG. 23e, the ear-worn structure 70 is arranged on the eyeglass structure 72 via the engagement structure 204, and in particular, the engagement structure is configured to have a bending portion toward the location of the occipital lobe at the rear of the head. Accordingly, in this embodiment, the electrode 721 on the engagement structure is configured to be of the scattered type in order to facilitate the electrodes to penetrate through hairs and contact with the scalp. As for the other electrode 702, it is arranged on the surface of the ear-worn structure in order to contact with the ear. With such configuration, the electrode 702 arranged on the ear-worn structure can be regarded as the reference electrode, and electrode 721 on the engagement structure can be regarded as the activity detection electrode, so as to acquire EEG signals from the occipital lobe. In this embodiment, the circuits can be located in the engagement structure and/or the ear-worn structure without limitation. In addition, the engagement structure can be configured to attach onto the temple, or to replace a portion of the temple, without limitation.

Furthermore, the electrical connection between the electrodes disposed on the eyeglass structure and the circuits can also have different possibilities. For example, the electrical connection can be achieved directly by a eyeglass structure which is made of a conductive material. Alternatively, it can also be configured to arrange a conductive portion on the eyeglass structure. Both are feasible methods.

Since the eyeglass structure is able to provide greater selection of contact locations with the head, such as the location around the nose or at the rear of the head, when the ear-worn structure and the eyeglass structure are used in conjunction with each other, the physiological signals capable of being acquired are of broader scope; therefore, it is of great advantages.

Furthermore, as shown in FIG. 25b, the circuits can also be located in a wrist-worn structure. Similar to the condition mentioned above, users can wear the wrist-worn structure, such as watch and wristband, equipped with EEG signals acquiring function on the wrist normally. And, when there is a need to measure EEG signals, it can be further connected to the EEG electrodes configured in an eyeglass form. Alternatively, the wrist-worn structure and the eyeglasses can be worn normally and when there is a need for measurement, it only needs to complete the connection therebetween. Therefore, such embodiment is also very convenient and can be integrated into daily life. Here, the eyeglass structure for carrying the electrodes and for connecting with the wrist-worn structure can anyone mentioned above without limitation.

In addition to arrange the EEG electrodes on the ear-worn structure and the eyeglass structure, the brain activity sensor of the present invention can also be configured to have other type of EEG electrodes. For example, electrodes can be extended from the ear-worn structure or the eyeglass structure for arranging onto the head or other locations, e.g., the forehead for acquiring EEG signals from the frontal lobe, the top of the head for acquiring EEG signals from the parietal lobe, and/or the rear of the head for acquiring EEG signals from the occipital lobe. Particularly, when it is configured to be the eyeglass type, the electrode at the rear of the head can also be achieved through extending the temple toward the rear of the head. Consequently, therefore is no limitation. Furthermore, when the mounting location of the electrode contains hairs, such as at the top and the rear of the head, it can be selected to use electrodes capable of penetrating through the hairs, e.g., pin type electrode, scattered electrodes or other type of electrode; alternatively, the electrode carried on a spring as mentioned above can also be used for increasing the usage convenience.

It shall be noted that the aforementioned preferred embodiments are provided for illustration purpose only, and the present invention shall not be limited to such embodiments, and the embodiments can also be modified and/or different embodiments can be combined with each other, which are all within the scope of the present invention.

Since the brain activity sensor according to the present invention uses ear(s) as the medium for installation, it is suitable to be integrated with an earphone. For example, it can integrate with an earphone which is used for listening music or a headset which is used for receiving and transmitting sounds, and further, it is not limited to the dual ear-worn type or single ear-worn type, or not limited to the in-ear housing or ear hook, which are all applicable under the concept of the present invention. Accordingly, it can be further integrated into the daily lives of users, for example, can be used during transportation. In addition, the type of earphone can also be selected according to the habit of users; therefore, the present invention is of great convenience.

Furthermore, when it is configured to be the eyeglass type, then it can use the eyeglass structure to install the sound production element and/or the sound receiving element (such as microphone), so as to provide the function of earphone and/or microphone, or alternatively, it can also extend an earphone from the temple of the eyeglasses. In such method, particularly, the sound production element and the earphone used can be the common air conduction type, or can be the bond conduction type, for example, a bond conducting speaker can be installed at the location where the temple contacts with the skull, or a bone conducting earphone can be extended form the temple, without limitation.

The brain activity sensor according to the present invention can also be configured to communicate with a portable electronic device, such as, to communicate with an external electronic device, e.g., a smart phone, a tablet etc., via wired or wireless connection, e.g., an earphone jack, Bluetooth etc. Consequently, when equipped with the sound production element (air conduction type or bone conduction type), an ear-worn or eyelgass brain activity sensor of the present invention can be used for hands-free voice communication, and also for listening music from the portable electronic device. Besides, through mounting vibration module, sound production element (air conduction type or bone conduction type), display element and light emitting element, the ear-worn and/or eyelgass brain activity sensor of the present invention can be further configured to be an information providing interface for the portable electronic device, for example, it can be used to provide incoming call alert, mobile phone message notice etc. so as to further integrate into the daily lives of users. As to the method for providing information, it can be achieved with many possibilities, including but not limited, the sound, the vibration, the light emission, lens display and/or so on, without limitation.

Moreover, when it is configured to include the earphone function, particularly when it is used for listening music, it is preferable to employ the dual ear-worn configuration so as to provide better sound effect for users. For example, two auricles both can have an in-ear housing mounted thereon, and through a wireless or wired connection therebetween, the music can be provided, such that the music can be stereo with left and right tracks. In addition, it can also be configured in such a way that the earphone includes a memory for storing music and is provided with the playing function, as a result, it is able to play music for listening without communication with the portable electronic device, which is even more convenient.

In a preferred embodiment, a single-ear brain activity sensing device of the present invention is configured to include a wireless transmission module, such as Bluetooth, in order to communicate with an external portable electronic device, for example, the physiological signals and information acquired can be wirelessly transmitted to the portable electronic device for further providing to the user.

Furthermore, in addition to the function related to physiological signal acquisition, the single-ear brain activity sensing device of the present invention can also be equipped with the sound production element and an electrical signal transmission port for receiving external signals, such as audio signal. Here, the audio signal can come from different sources. For example, it can come from another ear-worn device connected to the electrical signal transmission port, such as the audio signal saved in said another ear-worn device. Alternatively, it also can come from the external portable electronic device via a wired or wireless connection; for example, the audio signal from the external portable electronic device can be transmitted to another ear-worn device in a wire or wireless manner, and through said another ear-worn device connects to the electrical signal transmission port, the audio signal can be further transmitted to the single-ear brain activity sensing device; or the audio signal also can be transmitted to the single-ear brain activity sensing device through a wired connection from the portable electronic device to the electrical signal transmission port. All of the above are all possible choices.

As for the playing of the audio signal, it is executed by an audio control circuit. In an embodiment, the audio control circuit is located in another ear-worn device, wherein through the electrical connection between the electrical signal transmission ports of the single-ear brain activity sensing device and said another ear-worn device, the audio control circuit is able to drive the sound production element in the single-ear brain activity sensing device to play the audio signal. Further, if said another ear-worn device is also equipped with the sound production element, then stereo sounds can be achieved.

Accordingly, with the design of the physiological signal acquiring circuit and the audio control circuit are separately arranged in two ear-worn devices, advantageously, the connection between the two ear-worn devices can be configured to be removable. Therefore, for example, when a user wishes to only perform the physiological signal acquisition, said another ear-worn device can be removed; whereas when there is a need to listen to music, then the user can then connect back said another ear-worn device (and connect with the portable electronic device). Consequently, it is very convenient for use. Moreover, said another ear-worn device can also be used independently for providing music at single ear. Further, if said another ear-worn device is also equipped with the sound receiving element, then said another ear-worn device can be independently used as a headset of the portable electronic device. In addition, said another ear-worn device can also be configured to have electrode(s) mounted thereon, so as to allow the two ear-worn devices to together perform EEG signal acquisition. Under such condition, the connection between the two ear-worn devices can be used for transmitting the audio signal and can also be used for transmitting physiological signals.

Accordingly, through such design, the two ear-worn devices can be used in combination and used independently, which are able to cope with different usage demands of users.

It shall be noted that based on different usage purposes and design requirements, the transmissions between two ear-worn devices, including the transmission of audio signal and transmission of physiological signals, also have various possible combinations. For example, under the condition where one single ear is able to acquire the physiological signals, a wired connection between the two devices can be used to transmit audio signal only. Alternatively, if the acquisition of physiological signals requires electrodes mounted on both the ear-worn devices, then a wired the transmission should be employed, and under such condition, the audio signal can be transmitted in a wired or wireless manner, without limitation.

As the operation interface used for controlling the audio playing and wireless connection, it can be located at a location convenient to the user, such as located on the connecting wire between one of the ear-worn devices and the portable electronic device or on the connecting wire between two ear-worn devices, or integrated with the external housing, without limitation.

Consequently, when two ear-worn devices are used, no matter the connection therebetween is achieved in a wired or wireless manner, the following choices are available for the control of the audio playing and the physiological signal acquisition. For example, it can be configured that the circuits at one of the ear-worn devices controls the physiological signal acquisition, and the circuits at the other controls the playing of sounds. Alternatively, it can also be configured in such a way that the circuits at one of the ear-worn devices controls both the physiological signal acquisition and the playing of sounds. Thus, there is no limitation. Furthermore, regarding the configuration of electrodes, it can be configured that both the ear-worn devices have the electrode mounted thereon, for example, the electrodes at two devices can cooperate jointly to acquire EEG signals, or two devices can perform the EEG signal acquisition independently, or changes can be made through setting based on different needs. Therefore, the present invention is not limited to any specific configurations.

Moreover, the brain activity sensing device of the present invention can also be configured to be equipped with a connection structure for function expansion. As shown in FIG. 26a, a connection structure 80 can be configured to protrude downward from the in-ear housing. In addition, FIG. 26b shows that the connection structure 80 protrudes outward and extends to the auricle backside. Alternatively, it can also be configured as the one shown in FIG. 26c. Therefore, various choices can be made depending upon the actual needs without limitation.

Such connection structure further provides more possibilities. For example, in a preferred embodiment, the connection structure can be used for connecting one of the electrodes for acquiring EEG signals, such as, FIG. 26b shows that the electrode 82 is directly connected to the connection structure 80 in order to contact with the auricle backside. Furthermore, FIG. 26c shows that the electrode 82 is arranged on an external member 84 which is connected to the connection structure 80 so as to contact the V-shaped area. Under such condition, it only needs to cooperate with the electrode on the in-ear housing, and then EEG signals can be acquired. Alternatively, it can also be configured to use a connecting wire to connect the electrode to the connection structure, so that the electrode can be arranged at other location, such as, the other ear, or the head. As for the installation medium, there are numerous choices, such as another ear-worn structure, eyeglass structure, head-mount structure or electrode patch are all feasible, without limitation. Here, when the electrode can be arranged on the head, advantageously, the sampling locations for acquiring EEG signals are also increased, and it is able to acquire EEG signals from different cerebral cortices.

In other words, the connection structure provides the possibility to allow the electrode to extend out of the in-ear housing. In addition, it can further use a carrier to achieve the installation of electrode, wherein the carrier can be, as mentioned previously, the external member 84, another ear-worn structure, eyeglass structure, head-mount structure or electrode patch, without limitation.

Furthermore, for example, under the condition where the in-ear housing includes two electrodes, if the electrode on the in-ear housing cannot achieve a stable contact, then the connection structure can be used as an additional contact choice for improving the contact stability. Namely, the externally connected electrode 82 can be used to replace the electrode on the in-ear housing which cannot contact stably. And, the aforementioned various embodiments, e.g., directly connecting with the electrode, or connecting via the carrier carrying the electrode, are all applicable.

Moreover, the connection structure can also be used for the expansion of other functions. For example, it can be used for charging, and/or under the condition where the sound production element is equipped, it can be used for connecting to the sound production element of the other ear-worn device so as to provide stereo sounds. Thus, there are various possibilities. Furthermore, as mentioned above, the arranging location and protruding direction of the connection structure can also be modified according to the needs, such as extending in a downward direction, extending to the rear of the ear, or extending toward the direction of the face, and so on. Therefore, it is not limited to any specific configurations thereof.

Furthermore, other than performing EEG signal acquisition, the brain activity sensor of the present invention is also able to include other physiological sensing elements or electrodes for acquiring other physiological signals.

For example, it can also include at least one pair of light emitting element and light receiving element. Here, the light emitting element and the light receiving element refer to sensing elements which acquire light signals based on photoplethysmography (PPG) principle, e.g., measurements performed by penetration or reflection method, so as to acquire the physiological information of blood. And, the information can be used for further analysis to obtain other physiological information, for example, the variation of blood oxygen saturation can be obtained, and by analyzing the serial pulse variations, the heart rate sequence can be obtained for performing other relevant analyses. Therefore, the application scope is extremely wide without limitation.

When it is configured to be the ear-worn type, the light emitting element and the light receiving element can be located at the surface contacting with the ear or the skull, such as earlobe, ear canal, ear canal opening, tragus, intertragal notch, antitragus, concha wall, concha floor, auricle backside, V-shaped recess, or the skull skin at the intersection between the auricle and the skull without limitation. Therefore, any locations of the inner or outer side of the auricle and/or adjacent to the auricle contacted by the ear-worn structure are all feasible. One advantageous method is to contact the ear canal opening or the floor of cavum conchae/cymba conchae, which is particularly suitable to employ the in-ear housing along with the electrode(s) located on the surface of the in-ear housing. Another suitable configuration for the in-ear housing is to configure the light emitting element and the light receiving element to contact the tragus and/or the intertragal notch, for example, FIG. 21 shows that the light emitting element 210 and the light receiving element 212 along with the electrode 100 are jointly installed on the surface of the portion of the in-ear housing not entering into the ear canal, and when the setting of the portion of the in-ear housing entering into the ear canal is completed and the light emitting element and the receiving element are aimed at the tragus, it is able to obtain the physiological information of blood from the tragus. Under such condition, the in-ear housing can further provide the function of light shielding, which is even more beneficial for obtaining high quality signals. In addition, only one wearing action is enough to achieve the installation of the electrode as well as the light emitting element and the light receiving element, which provides a very convenient choice.

Moreover, when it is configured to be of the eyeglass type, the light emitting element and light receiving element can be located at any location of the eyeglass structure capable of contacting with the skull and the ear, such as nasal bridge, area between two eyes, temples, auricles, area adjacent to auricles, without limitation. For example, the light emitting element and the light receiving element can be installed on the temple together with the electrode in order to contact the V-shaped recess, the upper portion of the auricle backside, and/or the skull adjacent to the auricle, such as temple. In addition, it can be further configured as the electrode surrounding the light emitting element and the light receiving. As a result, the contact locations can be simplified and the usage complexity can be reduced.

Furthermore, when it is configured to be the type as shown in FIGS. 24a-24c, the light emitting element and the light receiving element can be arranged at the inner surface of the wearable structure as being worn on the head, so as to acquire physiological signals of blood from the head, such as, in addition to the blood oxygen saturation and heart rate sequence, the variation of blood flow in the brain also can be obtained which can represent the status of brain activity. Alternatively, the light emitting element and the light receiving element also can be installed at the location where can be accessed by the hand as the wearable structure is mounted on the head or the neck, for example, it can be exposed on the surface, so that the blood physiological signals can be obtained from the hand. Furthermore, the light emitting element and the light receiving element can also be arranged at the surface of the ear-worn structure or the eyeglass structure for being accessed by the hand of the user, thereby obtaining the blood physiological signal from the hand.

In addition, ECG electrodes also can be included for acquiring ECG signals, such as at least a first ECG electrode and a second ECG electrode, wherein the first ECG electrode can be configured to contact the user's auricle or the skull when the brain activity sensor of the present invention is worn on the user. When utilizing the ear-worn structure, for example, where the extension member contacts the V-shaped recess, the auricle backside or the skull, and where the in-ear housing contacts the inner side of the auricle can be used to mount the first ECG electrode. Alternatively, when utilizing the eyeglass structure, where the temple contacts the V-shaped recess, the temple, the auricle backside, the skull skin adjacent to the auricle, and where the nose pad contacts the nasal bridge, the naison, and the area between two eyes can be used to mount the first ECG electrode.

Regarding the second ECG electrode, there are various possibilities. For example, it can be arranged on an exposed surface of the ear-worn structure, the eyeglass structure (or the engagement structure), for being touched by the user's hand. In other words, the user only needs to raise his/her hand to touch the electrode during the measurement, and the ECG signals can be obtained, which is extremely convenient. Here, the exposed electrode can be made of metal, conductive rubber or any conductive material, without limitations. In addition, the electrode can be further configured to be non-contact electrode, such as capacitive electrode, inductive electrode, or electromagnetic electrode so as to increase the use convenience. Moreover, it can also use a connecting wire to extend the electrode outward for installing at other location, such as, the neck, the shoulder, the chest, the upper arm, the wrist, the finger and so on. Here, particularly, a wearable structure can be further employed to achieve the installation of the second ECG electrode, such as, a neck-worn structure, a shoulder-worn structure, an arm-worn structure, a wrist-worn structure, a finger-worn structure, or a patch, which all facilitate the securement of the electrode. It is advantageous that since two electrodes are both secured on the user's body via wearable structures, continuous ECG signal acquisition can be achieved. When a memory is provided, it is able to record the heart activity of the user for a long period of time, which is extremely helpful for the physician in diagnosis. It shall be noted that even the ECG electrodes are installed through the wearable structures, the ECG signals still can be acquired as necessary, without limitation, and the user can select according to the actual needs.

In addition, when the configurations in FIGS. 24a-24c are employed, the first ECG electrode similarly can be arranged on the ear-worn structure, and the second ECG electrode can be arranged on the location of the wearable structure accessible by the hand. In this embodiment, the touched by the hand can be performed as the wearable structure is worn on the neck or on the head, and in both ways, ECG signal can be obtained. Moreover, similarly, it can also use a connecting wire to extend the electrode outward without limitation.

It shall be noted that both ears can be selected as the location for the installing the ECG electrode. However, after experiments, it is learned that the contact location of the exposed electrode or the extended electrode can affect the signal quality, wherein when the left upper limb touches the exposed electrode, or the extended electrode is arranged on the left upper limb, the quality of ECG signals obtained is far better than the signal obtained from contacting the right upper limb. Particularly, when the electrodes are implemented to contact the left ear and the left upper limb respectively, an optimal signal quality can be obtained. Therefore, when acquiring ECG signals via contacting the ear, it is preferable to use the left upper limit to contact the exposed electrode or the extended electrode, so as to prevent the occurrence of poor signal quality which might lead to errors during analysis.

Furthermore, the first ECG electrode which contacts the auricle or the skull skin can also be shared to acquire EEG signals. In other words, one of the electrodes on the ear-worn structure or the eyeglass structure can be used as the EEG electrode and the ECG electrode at the same time. Accordingly, the manufacturing cost and complexity can be reduced, and the location required for contact can also be reduced, thereby increasing the convenience. Moreover, the second ECG electrode can also be shared to acquire other physiological signals, for example, it can be an electrode extended from the EEG electrode to the exposed surface, or it can be a portion of an EEG electrode which is formed to continuously distribute from the inner side to the outer side. Since ECG signals (falls within the range of mV) and EEG signals (at the range of several to tenths μV) are of significant amplitude differences, it is able to separate two signals without affecting the analysis thereof even in the case of sharing use.

Certainly, it can also be configured to equip with the light emitting element and the light receiving element as well as the ECG electrodes at the same time. Under this condition, the time required for the pulse wave to propagate from the heart to the detection location of the light emitting element and the light receiving element can be obtained, which is known as the Pulse Transit Time (PTT). And, since PTT is related to the arterial stiffness affecting the level of blood pressure, the specific relationship between PTT and the blood pressure value can be used to calculate the reference blood pressure value.

Furthermore, when PTT is obtained through EEG signals which is acquired by employing the hand to touch the second ECG electrode on the exposed surface, since the hand needs to be raised to the position of the exposed electrode, under such condition, no matter the detection location of the light emitting element and the light receiving element is at the auricle inner side or backside, the skull adjacent to the auricle, the nasal bridge/naison/area between two eyes or the hand touching the exposed electrode, the height thereof relative to the heart can remain unchanged. Based on haemodynamics, it is known that PPT might be affected by the measurement location and the height difference from the heart. Therefore, through such method, the impacts caused by non-fixed sampling location relative to the heart which is commonly seen in the conventional PPT measurement can be eliminated. Accordingly, after calibration, it is able to stably obtain precise blood pressure values. In addition, such measurement method is also not affected by the posture during measurement, such as standing or sitting, which is of great advantages.

Followings are the applicable fields of the monitoring device with the brain activity sensor of the present invention.

One of the applicable fields is neurofeedback. For example, when the purpose of neurofeedback procedure is relaxation, one possibility is to observe the percentage of α wave among brain waves. Generally, when α wave (around 8-12 Hz) is dominate, human body will be in a relax and wake state, so that through observing the percentage of α wave can be referred to the relaxation degree. Alternatively, when the purpose is to improve focus, it can be selected to observe the percentage of θ wave (around 4-7 Hz) and β wave (around 12-28 HZ). Among brain waves, dominate β wave means human body is in a wake and nervous state, and dominate θ wave means human body is in a relax and unconscious state. Therefore, through increasing the ratio of β wave to θ wave, it will be able to achieve a purpose of focus improving. For example, one method to cure ADHD (Attention deficit hyperactivity disorder) is to observe the ratio of θ wave/β wave in the neurofeedback procedure. Besides, SCP (slow cortical potential) is also one kind of brain activity that will be observed in the neurofeedback procedure for improving focus, wherein the negative shift of SCP is related to more focused concentration, and the positive shift of SCP is related to reduced concentration. Also, other brain waves can be used, for example, the occurrence of γ wave (around 28-40 Hz) means the human body is in a highly focused state. Further, neurofeedback also can be used to monitor the occurrence of epilepsy for further diagnosis. Thus, there is no limitation.

Moreover, because the relaxation degree of human body also can be determined via the ANS (Autonomic Nervous System) activity, e.g., when the PNS (Parasympathetic Nervous System) activity increases and/or a ratio of PNS/ANS (Sympathetic Nervous System) activity increases, it means the relaxation degree is increased, if the device of the present invention is configured to equip the light emitting element and the light receiving element and/or ECG electrodes, then through analyzing heart rate sequence and thus HRV, it will be able to obtain the ANS activity. Therefore, this ANS activity information and the information of brain activity can be combined to evaluate the relaxation degree for neurofeedback.

As to how to provide the physiological information to the user in real time so as to achieve the effect of neurofeedback, there is no limitation. For example, if it is implemented as earphone type, the information can be provided as audio type, e.g., as the brain wave shows the human body is in a nervous state, a rapid music can be used to represent it, and as in a relax state, a slow music can be used; or a powerful music can be used to represent a focused state; or it also can use the frequency of sound or voice to inform the user the physiological state; or a vibration at the portion that contacts the skin also can be used, e.g., use high and low vibration frequency to represent nervous and relax. Alternatively, the glasses also can be employed to provide information in visual type. Therefore, the information can be provided by the ear-worn structure or glasses structure with visual, audio and/or tactile sensing signals without limitation.

Further, the information also can be provided by a device that is connected with the monitoring device, e.g., a smart phone, a sound production device, or an illumination device.

Another applicable field is to help for breath training. Based that RSA (Respiratory Sinus Arrhythmia) information can be obtained through heart rate sequence, the synchronization among heart rate, breath and EEG signal also can be used as the basis in the feedback procedure. According to researches, inhalations and exhalations will cause fluctuations of blood volume in blood vessel, and the fluctuations will transported to brain with blood flows, so as to cause the brain wave to have fluctuations in a low frequency section, e.g., lower than 0.5 Hz. Thus, through observing brain waves, it will be also possible to reveal respiratory pattern. Further, the sinus node and the vascular system are modulated by ANS, and ANS also will feedback the changes of heart rate and blood pressure to the brain via the baroreceptor system and thus influence the function and operation of brain, e.g., influence cerebral cortex, which can be detected by EEG and plus, a conscious control of breathing will also change the heart rate via ANS. Therefore, these three factors mutually influence one another. Accordingly, the good synchronization among these three facts means human body is under a relax state, so that the analysis result of synchronization can be used to provide to the user as the basis of self-regulation in the neurofeedback procedure.

Moreover, because enhancing the amplitude of RSA is beneficial to trigger relaxation response and relief accumulated stress and thus increasing the ratio of PNS/SNS activity, through observing the pattern of heart rate variation and guiding the user to inhalation at the time the heart rate starts to increase and to exhalation at the time the heart rate starts to decrease will be able to achieve the effect of enhancing RSA amplitude, which means to enhance the coherence between respiration and heart rate will also be able to achieve relaxation. Further, the amplitude between the peak and valley of RSA, namely, the delta of maximum and minimum in one respiration cycle, is related to the activity level of ANS, so that this information also can be provided to the user to be the basis of self regulation.

Furthermore, it also can be implemented to obtain the respiratory pattern by observing the fluctuation of blood flow, e.g., via positioning the light emitting element and the light receiving element at the ear or the forehead to acquire pulse variations, the variation of blood flow can be obtained.

Identically, the breath guiding/real time physiological information can be provided by the ear-worn structure or glasses structure via audio, visual and/or tactile sensing signal, or via the connected information providing interface, which can be changed depending on practical demands without limitation.

Here, particularly, the operation of the wrist-worn EEG monitoring device as shown in FIGS. 25a-25b to be applied in biofeedback and breath training is described. The wrist-worn device with the (one-side or dual-side) ear-worn structure provides portability to acquire EEG signals, so that the user almost can perform the biofeedback/breath training without time and location limitation. If ECG electrode(s) can be further mounted on the wrist structure to acquired ECG signals by cooperating with the electrode(s) on ear-worn structure, or the light emitting element and the light receiving element can be mounted on the ear-worn structure or the wrist-worn structure to acquire heart rate, it will be able to realize the respiration pattern so as to perform breath training. Plus, if ECG electrode(s) as well as the light emitting element and the light receiving element are equipped at the same time, it will be able to obtain PTT (Pulse Transit Time), and through the relationship between PTT and blood pressure, referential values of blood pressure can be calculated, or the PTT information also can be provided for feedback procedure. Therefore, the user only needs to wear the wrist-worn structure and the ear-worn structure, and then plural physiological information can be obtained. The operation is simple and convenient.

Furthermore, other than the situations described above, the wrist-worn structure also can be used to monitor other kinds of physiological signals. For example, while one electrode is mounted for contacting the wrist wearing the wrist-worn structure, another electrode also can be mounted on the wrist-worn structure for contacting another limb, so that ECG signals via two limbs can be acquired. Alternatively, two electrodes can be mounted for contacting the wrist wearing the wrist-worn structure, so that EDA signals and/or EMG signals can be acquired. Alternatively, a finger-worn structure can be further extended from the wrist-worn structure to bear electrode(s), so that, one possibility is two electrodes contacting the finger wearing the finger-worn structure to acquire EDA signals and/or EMG signals, and another possibility is one electrode contacting the finger wearing the finger-worn structure and another electrode, e.g., mounted on the wrist-worn structure, the finger-worn structure, or the glasses structure, to contact another limb so as to acquire ECG signals. Besides, it also can be configured to equip the light emitting element and the light receiving element for obtaining blood-related physiological information, e.g., heart rate and oxygen saturation.

Moreover, because the position of wrist-worn structure is just as the general position for mounting information providing interface, e.g., watch or wrist band, during biofeedback or breath training, it will be very nature to provide the feedback information and/or breath guiding via the wrist-worn structure and/or to be used as user's input interface, which is convenient. Besides, if the user selects to close eyes in the biofeedback or breath training procedure, the feedback information and/or guiding also can be provided via the sound production element in the ear-worn structure, or via vibrations generated on the ear-worn structure and/or wrist-worn structure.

Additionally, the audio and visual sensing signals provided through the ear-worn structure and/or glasses structure also have other applications, such as, through the variations of sounds or lights, it will be able to lead the brain to achieve a state of coherence, synchronize or entrainment, or it also will be able to stimulate the brain and through monitoring the responses in brain by the physiological monitoring device, the condition of brain can be revealed.

Another application is to monitor the physiological condition for notification, for example, it can be used to monitor alertness and drowsiness. As described above, by observing the frequency variation of brain wave, it will be able to reveal the brain state. Thus, based on this, the notification can be executed. As adopting ear-worn structure, if the electrode is located at the front of the ear or near the temple, or as adopting glasses structure, if the electrode is located on the nose pad or near the ear, EOG signals can be acquired. Then, through analyzing EOG signals, information such as blinking frequency and speed can be obtained, and thus, the user's consciousness, drowsiness, and/or fatigue can be revealed. And, since the brain activity sensor of the present invention no matter in ear-worn type or glasses type is suitable for wearing, especially when driving, it only needs to produce sounds or vibrations from the ear-worn structure or flashes from the glasses structure, or to produce notification from the device connected therewith such as smart phone, so that the purposes of improving alertness and preventing drowsiness can be easily achieved. Accordingly, the possibility to cause traffic accident can be effectively reduced, which is practical and important.

Moreover, the device of the present invention also can be applied to acquire sleep related information. As known, EEG signals are the main basis for deciding sleep stage. Generally, the conventional measuring settings is, for example, to put multiple electrodes on the scalp while each electrode connects with a cable connecting to a machine, and obviously, this kind of settings is either comfortable nor convenient for the user. In this invention, the deposition of electrodes is easily and conveniently accomplished by wearing the ear-worn structure or the glasses structure, so that when being used during sleep, the user can feel fewer burden and more nature, thereby the measuring results would be closer to the regular sleep condition.

Further, through increasing electrode(s) or through sharing electrode(s), it also can be used to measure other electrical physiological signals, e.g., EOG signals, EMG signals, ECG signals, EDA signals etc. and all these are included in PSG (Polysomnography). For example, EOG signals can provide information of REM (Rapid Eye Movement), EMG signals can provide information of sleep onset, sleep offset, bruxism, and REM, ECG signals can be used to realize the physiological condition during sleep, e.g., ANS activity and heart activity, and EDA signals can provide information of sleep stage. And, if the light emitting element and the light receiving element are further equipped, oxygen saturation can be acquired to realize if hypopnea is happened; by equipping movement sensing element, such as, accelerometer, G sensor, gyroscope, and magnetic sensor, the information of body movement also can be obtained; and by equipping microphone, the snoring condition also can be revealed. Therefore, only through this simple sensor on the ear, multiple sleep information can be provided, which is extremely convenient.

Another application is to use for evoked potential test. According to the positions of the active electrodes, the measured EEG signals are from temporal lobe which is near the ear. The temporal lobe is involved in primary auditory perception, and is also related to language and memory. Therefore, through evoked potential test, for example, it is able to reveal the user's response to audio stimulations, such as, response speed, response level (the amplitude of responded brain wave), and adaptation (via continuous audio stimulations), and in accordance with the device structure of the present invention, it is also able to know respective response conditions of left side temporal lobe and right side temporal lobe.

Further another application is to apply stimulation to human body, so as to achieve effects of changing physiological condition, brain state, and/or consciousness. For example, the common usage is to achieve relaxation, to improve concentration, such as, cure ADHA (Attention deficit hyperactivity disorder), to enhance memory, to change mental condition, such as cure PTSD (Post traumatic Stress Disorder), to enhance mental capability and performance, such as cure melancholia, to change brain state, such as cure dementia, to change cognitive state, and to change/induce sleep state etc.

In this application, the advantage of ear-worn structure is the deposition position thereof is ear, so that it only needs to equip sound production element (air conduction or bone conduction), and then auditory stimulation can be provided, or to equip vibration module, and then tactile stimulation can be provided. As to visual stimulation, it can be achieved by employing a display element extended from the ear-worn structure and located within the user's sight, or further, by cooperating with the glasses structure so as to use the lens to display, for example, by projecting, or by mounting the displaying element, e.g., LED, LCD or other type displaying element, on the lens, e.g., at one side or both sides of the frame or the temple, so as to produce flashes and/or color variations. In addition, while both ear-worn structure and glasses structure are employed, it is also possible to provide the audio and tactile stimulations from the glasses structure, such as, the sound production element (air conduction or bone conduction) can be deposited on the temple at a location near the ear, or the vibration module can be deposited on the frame or temple at a location adjacent to the head. Thus, there is no limitation. Further, the electrodes also can be used to produce electrical stimulation.

First, since the ear-worn structure/glasses structure of the present invention is originally equipped with electrode(s), it will be advantageous to perform electrical stimulation.

The common electrical stimulation includes tCS (transcranial Current Stimulation), TENS (Transcutaneous Electrical Nerve Stimulation), MET (Microcurrent Electrical Therapy), and other known electrical stimulations, wherein the common used tCS includes tDCS (transcranial Direct Current Stimulation), tACS (transcranial Alternating Current Stimulation) and tRNS (transcranial Random Noise Stimulation). Among these, particularly, tCS is to apply a weak current (which is usually lower than 2 mA) to the local tissue above the cerebral cortex for influencing the activity of cerebral cortex, and during operation, usually, the user won't feel the current. As known, different regions of cerebral cortex correspond to different body functions, as shown in FIG. 1, such as, occipital lobe is concerned with visual process, temporal lobe is concerned with the understanding of speech, parietal lobe is concerned with the reception and correlation of sensory information, and frontal lobe is concerned with behavior, learning, personality, and voluntary movement. Therefore, by locating the electrodes at the skull corresponding to different cortex region, it is able to not only acquire the activity of different cortex regions, but also produce electrical stimulation for achieve an influence on the local cortex.

Another kind of electrical stimulation is electrical stimulation of tongue. Research shows that electrical stimulation of the tongue stimulates two major cranial nerves: the lingual nerve (part of the trigeminal nerve) and the chorda tympani (part of the facial nerve). The electrical stimulation of the cranial nerves creates a flow of neural impulses that are then delivered to the parietal lobe and directly into the brain stem, which is the main control center for many life functions, including sensory perception and movement. From the brain stem, these impulses travel throughout the brain and activate or reactivate neurons and structures involved in human function—the cerebral cortex, spinal cord and potentially the entire central nervous system. And, by utilizing an oral structure to carry the electrode(s), the electrical stimulation of tongue can be achieved. In practice, while the oral structure is implemented to carry plural electrodes, it is preferably to arrange the electrodes in a pattern, such as, matrix, for enhancing the effects.

Other than the effects described above, applying electrical stimulation to human body is also known contributive to change the physiological state and improve some syndromes, such as, local pains, like shoulder or neck pain, migraine, depression, epilepsy, and stroke. Generally, the locations for applying electrical stimulation are, for example, trigeminal nerve, vagus nerve, sympathetic nerve, or/and cerebral cortex, and the syndromes of sole muscles at shoulder and neck are also near the head, which are all adjacent to the locations for mounting the wearable structures of the present invention, such as, the war-worn structure, the eyeglass structure, the neck-worn structure, and/or the head-mount structure, and their contact positions, such as, the earlobe, the auricle, the ear canal, the skull near the auricle, the neck, the temple area, the forehead, the top of the head, and the rear of the head. For example, among the branches of trigeminal nerve, auriculotemporal nerve is located near and above the ear, and supraorbital nerve, supratrochlear artery nerve and ophthalmic nerve are located near the eye socket and the forehead, and all these are also the contact positions of the eyeglass structure/ear-worn structure as worn on the head/ear(s). Therefore, it is suitable to utilize the existing wearable structures of the present invention to execute the stimulation. In addition, applying electrical stimulation at acupuncture points is also able to improve the physiological state.

For example, it can be implemented to use the eyeglass structure and perform the electrical stimulation on the brain directly through the two electrodes thereon, such as, the electrodes contacting two sides of the head, or two electrodes respectively contacting the area between two eyes and one side of the head. Alternatively, it also can be implemented to use the ear-worn structure, so as to apply electrical stimulation on the brain through the electrodes on the in-ear housing and/or on the extension member as described above. Furthermore, it also can be implemented to use the electrodes on the neck-worn structure or the head-mount structure to apply the electrical stimulation, and the above mentioned neck-worn/head-mount structure is also suitable. In addition, it also can adopt two wearable structures, such as, the ear-worn structure with the head-mount structure, the ear-worn structure with the neck-worn structure, or the ear-worn structure with the eyeglass structure. Since the contact of electrodes are done right after the wearable structure is worn on, no matter employing which kind of wearable structure, all can make the electrical stimulation become easier and more convenient.

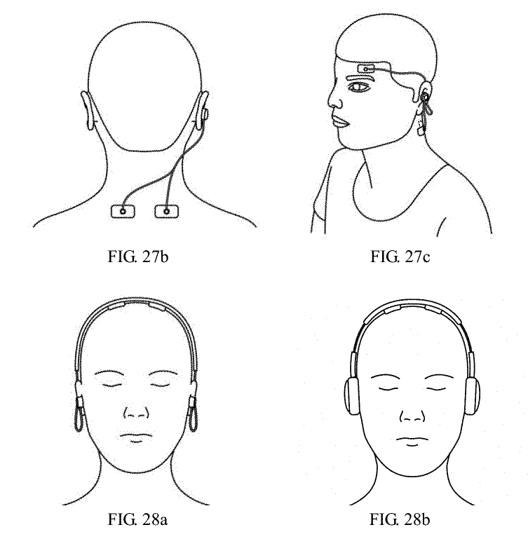

Other than directly utilizing the electrode(s) on the wearable structure to apply electrical stimulation, it also can have other choices. For example, electrode(s) can be extended from the wearable structure, such as, only one electrode is extended for cooperating with the electrode on the wearable structure to apply electrical stimulation, or two electrodes can be extended, without limitation. When the extended electrode is employed, advantageously, the contact range becomes wider and is not limited to where the wearable structure is located. As shown in FIG. 27a, the electrode extended from the eyeglass structure can contact the rear of neck, the area behind the ear, or the forehead etc. s shown in FIGS. 27b-27c, the electrodes extended from the ear-worn structure can contact the forehead, the temples, the rear of neck or the area behind the ear etc. And, similarly, the neck-worn structure and the head-mount structure also can be configured to have extended electrode for increasing range for applying electrical stimulation. It shall be noted that it can be implemented to extend one or two electrode without limitation.

When the electrode is extended, an attaching element can be used for attaching the electrode on the skin. For example, it can be the patch as shown in FIG. 27b, or can be belt for surrounding a body portion, or also can be another wearable structure, such as, an ear-worn structure, neck-worn structure, arm-worn structure, wrist-worn structure, finger-worn structure extended from the eyeglass structure, or an eyeglass structure, neck-worn structure, arm-worn structure, wrist-worn structure, finger-worn structure extended from the ear-worn structure, or an ear-worn structure, arm-worn structure, wrist-worn structure, finger-worn structure extended from the head-mount/neck-worn structure. All these are feasible without limitation. Alternatively, when two electrodes are extended, it can be implemented to use two attaching elements to carry two electrodes, or also can use one attaching element to carry two electrodes, without limitation.

It shall be noted no matter the electrode(s) is mounted on the wearable structure or extended out, it can be implemented to be dry electrode or wet electrode, such as, electrode with conductive gel, without limitation. It is especially advantageous is to adopt a self-adhesive electrode, such as, patch electrode, so as to further enhancing the contact stability between the electrode and the skin. As to how to utilize the wet electrode, there are many possibilities, for example, it can be configured to use wet electrode by employing extended wet electrode or by replacing the dry electrode on the wearable structure with wet electrode, without limitation.

When adopting dry electrode, it is advantageous that the contact assurance structure can be used to ensure the contact during the electrical stimulation, for example, the scattered contact points and/or shrinkable structure are especially suitable for where contains hairs, and/or has curve surface. Therefore, it can be selected to use the suitable type of electrode depending on the purpose without limitation.

In practice, a signal generation unit is employed to generate an electrical signal which is then transmitted to the electrodes connected thereto for applying electrical stimulation on the user. Accordingly, through altering the electrical signal, the electrical stimulation applied on the user can be changed. It shall be noted that electrical stimulation is non-invasive and the contents of the electrical signal can be different in accordance with different purpose. For example, it can be an electrical current or voltage variation based on sine wave, square wave or other waveform; or under a situation of adopting pulse wave, even with the frequency, it also can configured to change the stimulation period through pulse width modulation; or under a situation of adopting the direct current to apply electrical stimulation, the direct current can be used as offset and further load thereon a selected waveform. Therefore, all are feasible without limitation.

Furthermore, more advantageously, since the wearable structure of the present invention is originally designed to acquire EEG signals and/or other physiological signals, it is able to combine the physiological monitoring function and the electrical stimulation in one single device, and through which the effect of electrical stimulation can directly be confirmed with efficiency and convenience.

For example, one of the physiological states that will be changed by electrical stimulation is the state of brain activity. The brain activity can be revealed by measuring EEG signals, for example, as mentioned above, by observing the percentage of α wave and β wave, it is able to understand the relaxation or nervousness level of the user; further, through employing multi-channel measurement, it is able to recognize the activity and energy difference and the potential difference between left brain and right brain; in addition, by observing SCP, it is able to know the concentration degree. After realizing the state of brain activity, it can be used as the basis to adjust parameters of the electrical stimulation, such as, intensity, frequency, duty cycle, duration etc. so as to affect the brain and thus reach the purpose. Moreover, after applying electrical stimulation, it is able to know the stimulation effect by checking the state of brain activity and then to adjust again accordingly.

Alternatively, electrodermal activity (EDA) is also an indicator for observing the variation of physiological state. Through electrodes installed on the scalp or extended to install at other body portion, e.g., neck, shoulder, wrist, finger, it is able to acquire EDA signals. And, the checking of EDA can be performed before, during and/or after the electrical stimulation, so as to be the basis of determining and/or adjusting the pattern of electrical stimulation.

Alternatively, the variation of physiological state also can be observed through the variation of heart rate. After calculation, HRV (Heart Rate Variability) can be obtained from the heart rate, and HRV is known to be the best way to understand the ANS activity. Therefore, no matter the purpose of electrical stimulation is relaxation, concentration improvement, improvement of physical status, improvement of sleep state, change of brain status or treatment of some syndromes, through observing the variation of ANS activity, all these situations can be effectively controlled and thus being the basis to adjust the stimulation. Here, the obtaining of heart rate can be performed by light emitting element and light receiving element or by ECG electrode, without limitation. For example, the light emitting element and the light receiving element can be mounted at a location of the eyeglass structure or the ear-worn structure where contacts the skull skin and/or the ear, or at the extended patch, belt, neck-worn structure, head-mount structure, wrist structure, finger structure, all the feasible selections. In another aspect, if the heart rate is acquired through ECG electrodes, then the positions for installing ECG electrodes can be, for example, the skull/ear and the upper limb, two ears, and the neck/shoulder and the upper limb etc. Under such condition, the electrode(s) can be carried and stabilized by the wearable structure, patch or belt with convenience.

In another aspect, when the drowsiness is observed through detected brain waves, the electrical stimulation also can be used to notify the user or prevent the user to fall sleep, for example, the user can wear the eyeglass, earphone, neck-worn structure during driving or study, and through analyzing the detected brain waves to know if drowsiness occurs, the electrical stimulation can be applied based thereon.

It shall be noted, when the electrical physiological signals are detected, the electrode(s) for acquiring electrical physiological signals and for performing electrical stimulation can be implemented to have sharing usage, for example, sharing usage of one or two of the electrodes, which will further simply the entire configuration.

How to generate and adjust electrical stimulation based on the physiological state can be embodied in various ways. For example, it can be configured that the signal generation unit automatically control the generation of electrical stimulation, the pattern of electrical stimulation, and the parameters of electrical stimulation, such as, duration, current intensity, voltage, frequency, duty cycle etc.; or it also can be configured to be operated by the user, such as, the detected physiological information can be provided to the user via the mobile phone or the interface on the wrist-worn device, the eyeglass, the ear-worn device, and then, based thereon, the user can determine to perform the electrical stimulation or not, can select the pattern of electrical stimulation, and/or can decide to adjust the parameters or not. Certainly, it also can be implemented to change between auto-operation mode and manual operation mode in accordance with the actual needs without limitation.

In a preferred embodiment, a collection of electrical stimulation modes is provided for the user to have free selection; or it can be implemented that electrical stimulation modes that are related to the detected physiological information are picked out first and then provided to the user for selection; or it also can be implemented to allow the user adjusting the parameters as mentioned above. All these are feasible without limitation.

Consequently, performing the electrical stimulation through the wearable structure indeed provides an easier way for applying electrical stimulation, and with the real time detected physiological information of the user, it is able to improve the adjustment and selection of stimulation mode(s) and the achieved effects, so that this is indeed an advantageous embodiment.

In another aspect, under the condition that the ear-worn structure and/or eyeglass structure of the present invention is able to acquire EEG signals, particularly, it can further be applied to perform physiological resonance stimulation.

First, a brain activity detection unit is provided for acquiring EEG signals through two electrodes within a specific time period. Then, a processing unit is provided to perform a frequency domain analysis of the acquired EEG signals, e.g., through Fourier transform, or by utilizing digital filter, so as to obtain the energy distribution of EEG signals. Further, in each band, such as, δ band (0.1-3 Hz), θ band (4-7 Hz), slow α band (8-9 Hz), middle α band (9-12 Hz), fast α band (12-14 Hz), slow β band (12.5-16 Hz), middle β band (16.5-20 Hz), fast β band (20.5-28 Hz), and other bands, it is able to observe one or more peak energy, such as, an 8 Hz energy peak in α band, or an 8 Hz and a 10 Hz peak energy. And, after selecting the band, e.g., selecting α band, or self-defining a range of band, a stimulation signal generation unit can be employed to provide a physiological stimulation signal based on the frequency of the peak energy within the selected band, so as to apply to the user.

It shall be noted the brain activity detection unit has the function of brain activity sensor, sensing device, sensing system as described above and can be embodied by any one thereof, and for the purpose of simplification, the brain activity detection unit is employed as representation.

It shall be noted that the specific time period can be implemented as real time, e.g., to perform the frequency domain analysis every minute or shorter period of time; or the specific time period also can be implemented to be a longer period of time, e.g., every 5 minutes, and the time can be separated into several sections, so as to respectively perform the frequency domain analysis and then calculating the average, or the signals acquired during the whole period of time can be directly perform the frequency domain analysis. Thus, there is no limitation and can be changed in accordance with actual needs.

As to the frequency of the stimulation signal, after researches, it is preferable to be in proportional to the frequency of the peak energy. For example, if the frequency of stimulation signal is n, and the frequency of the peak energy is m, then as long as n and m are integers, any ratio thereof is feasible, such as, n:m can be 1:2, 1:3, 2:3, 3:2, 3:1 etc., without limitation. As a result, through this proportional relationship, it is facilitated for achieving entrainment therebetween, thereby further achieving resonance.

It shall be noted, in practice, the frequency of the peak energy and the proportional relationship that are determined by the method described above can be allowed to have minor deviations and be still in the scope of the present invention. Furthermore, it also can be implemented to mix plural stimulation signals in different ratios, such as, to mix one signal in a ratio of 1:2 with another signal in a ratio of 1:3, so as to form plural harmonic waves which can further facilitate the achievement of entrainment/resonance. In addition, when it is implemented to perform auditory stimulation, the stimulation signal can be mixed with music, such as, the sounds of nature, so that the user can accept more. Therefore, there is no limitation.

When the resonance is achieved, one possibility is the effect of increasing the target peak energy can be achieved, for example, the amplitude of the selected 8 Hz peak energy can be enhanced. Another possibility is the frequency of the selected peak energy within the selected band can be influenced, e.g., changed from 8 Hz to 9 Hz, in which the resonance therebetween causes a dragging force, thereby changing the frequency of the peak energy. As a result, through gradually increasing or decreasing the stimulation frequency, the dragging effect of changing the natural frequency can be achieved.

Furthermore, through the method of enhancing amplitude or changing frequency of the peak energy, it is possible to achieve the effect of changing physiological state, brain state, and/or consciousness, such as, to induce sleep, awareness, relaxation, meditation depth etc. In addition, it also can have positive effect on some brain related syndromes, such as, epilepsy, migraine.

As to the stimulation signals, there are many possibilities, such as, visual stimulation signals, auditory stimulation signals, or electrical stimulation signals. For example, the visual stimulation signals can be a visual signal with a flash frequency matching the proportional relationship, such as, flashes by LED, LCD or other displaying element. The auditory stimulation signals can be an auditory signal with a changing frequency matching the proportional relationship, such as, produced by the (air conduction or bone conduction) sound production element. In a special embodiment the auditory stimulation signals can be produced by two auditory sources, namely the so called binaural beats. Through providing two auditory signals have a frequency difference, which is proportional to the frequency of the peak energy, when two signals are input to the brain at the same time, the brain will feel like to receive a third auditory signal with said frequency difference. This kind of two auditory sources can be implemented by, for example, two sound production elements respectively located at two ear-worn structures, or at two temples of the eyeglass structure. Here, the eyeglass structure is especially suitable for employing bone conductive sound production elements, and in such way, the change of the eyeglass structure can be minimized. Alternatively, the sound production elements also can be implemented to extend from the eyeglass structure, such as, to extend two ear-worn structures from one temple, or respectively from two temples, which are all feasible.

The electrical stimulation also has various implemented ways. As mentioned above, through selecting different currents or voltages with different waveforms, the electrical stimulation mode can be changed. In addition, the body portion for applying the stimulation is also selectable, such as, as mentioned above, tCS, TENS, or electrical stimulation of tongue. There is no limitation.

Moreover, other than applying single stimulation, it also can be implemented to apply two stimulations at the same time, such as, visual stimulation plus auditory stimulation, or electrical stimulation plus auditory stimulation; or to simultaneously apply electrical stimulations at different cerebral cortices. There is no limitation. Furthermore, the second stimulation source also can be provided by an external device, such as, a light source, a sound production source, a mobile phone etc. Here, the frequencies of plural stimulation sources can be implemented to be identical or different without limitation, as long as they are in proportional to the frequency of the peak energy.

As performing the stimulation through resonance, with the detection of EEG signals, the effects of stimulation can be revealed during and/or after thereof, for example, if the target peak energy is increased pr not, and/or if the amplitude thereof is enhanced or not. Thereby, the stimulation performance can be changed promptly as the expected effects are not reached, for example, if the amplitude is enhanced not enough, it can promptly, such as, increase the strength of stimulation, increase the stimulation period, or change the waveform of stimulation signals, which are all helpful.

This kind of resonance stimulation is capable of precisely aiming at the existing frequency of brainwaves, so as to achieve the effect of enhancement, and also capable of providing real time adjustment, which is significantly effective.

Identically, no matter the type, the mode, or the parameters of the resonance stimulation, all can be configured to be selected by the user, such as, through the input interface on the ear-worn structure or eyeglass structure, e.g., button, touch control interface, light sensing, voice control, or through the external device communicated with the ear-worn structure or eyeglass structure, e.g., the operation interface of the smart phone or the wrist-worn device. Furthermore, the changes caused by the resonance stimulation also can be provided to the user through the information providing interface on the ear-worn structure or eyeglass structure or through the external device, such as, in an audio, visual, and/or tactile manner, so as to allow the user understanding the current physiological situation, which is also helpful for achieving the resonance.

In a special embodiment, as shown in FIGS. 28a-28b, the configuration of the head-mount structure (in a type of belt) located on the top of head with two ear-worn structures (in a type of earmuff or in-ear housing) is very suitable for acquiring EEG signals from the parietal lobe. As shown, when the ear-worn structure is implemented as in-ear housing, the connection with the head-mount structure can be achieved by connecting wire, and when the ear-worn structure is implemented as earmuff, it is mostly possible to form the head-mount structure and the ear-worn structures into one integration. However, it's not limited and can be implemented in other ways.

In practice, as shown, two electrodes 191, 192 are mounted on the head-mount structure at the locations corresponding to the parietal lobe, so as to acquire EEG signals. Alternatively, it also can be implemented to mount an electrode on the ear-worn structure as the reference electrode, so as to cooperate with the electrodes on the head for acquiring two channel EEG signals via referential montage. Alternatively, it also can be implemented to mount one electrode on the head-mount structure and another on the ear-worn structure, which is also able to acquire EEG signals from the parietal lobe. Furthermore, the electrode also can be located near the temporal lobe, such as, on the head-mount structure where near the ear, or on the ear-worn structure, so as to acquire EEG signals from the temporal lobe, and this is especially suitable for the earmuffs structure. Therefore, there is no limitation and can be varied in accordance with the actual needs. In addition, other than acquiring EEG signals, the electrodes also can be utilized to apply electrical stimulation, such as, tCS, resonance stimulation etc. Alternatively, it also can be implemented to carry the electrical electrode(s) for performing electrical stimulation through the attaching element(s), such as, the electrode(s) extended from the head-mount structure or the ear-worn structure. Here, for overcoming the contact problem caused by hairs on the contact location, preferably, the above mentioned contact assurance structure can be used, so that not only the electrode can penetrate hairs, but the contact range is also increased.

Since the structure described above is similar to the conventional headphone, it is also suitable to mount therein the sound production element (air conduction or bone conduction), as a result, it can be implemented to provide the auditory signal in a natural way, for example, to play the music stored therein, e.g., mp3 sound file, or to play the music from the external device; or it also can be implemented to provide related physiological information and/or operation information, e.g., all the audio stimulations above. In addition, since it is also able to mount sound production element at both ear-worn structures, under such condition, the stimulation by binaural beats also can be performed thereby.

Under this configuration, not only EEG signals can be acquired and/or the electrical stimulation can be performed, but the auditory signal also can be provided and/or the audio stimulation also can be performed, and plus, the headphone-typed configuration is a conventional style, which is highly accepted by the public. Therefore, it is advantageous.

Further, with this configuration, if the material made thereof can be selected to be soft and comfortable material, it is then suitable for being used during sleep. While sleeping, through detecting EEG signals, the brain activity status can be revealed, such as, REM (Rapid Eye Movement) stage, stage of deep sleep. Based on this, except that music which is capable of improving sleep can be provided, the various stimulations for the brain also can be determined, e.g., electrical stimulation and audio stimulation. As mentioned above, there are many kinds of stimulations applied on human body can have the effects of improving/inducing sleep, so that through this configuration, all these stimulations can be performed thereby naturally, which is advantageous. Further, other physiological sensing element(s) also can be mounted thereon for acquiring other physiological signals, for example, light emitting element and light receiving element can be used to acquire physiological signals of blood, so as to obtain the information about heart rate, respiration, oxygen saturation etc.; or other electrodes can be used to acquire, such as, EOG signals, EMG signals, EDA signals etc.; or microphone can be used to acquire sound signals from the user, e.g., the breathing pattern, snoring, sleep apnea etc., and all these are beneficial to understand more details about sleep. In addition, other than being the basis of adjusting physiological stimulation, the physiological signals also can be recorded for executing sleep analysis and diagnosis.

Moreover, it is preferable that the detection of EEG signals and/or other physiological signals also can be used as a basis before the electrical stimulation and/or resonance stimulation for determining whether the stimulation is executed or not, and/or which kind of stimulation should be executed.

If the purpose of stimulation is to relax, improve concentration, change mental status, change/induce sleep state, change brain state, such as, cognitive state, it can be implemented to observe the brainwaves or other physiological signals first for realizing if the physiological state is stable, and then, to decide if stimulation should be started, and/or which kind of stimulation is more suitable, thereby helping to achieve the stimulation effect more rapidly.

For example, through observing the brain waves, it is able to know the user is in a relaxed or nervous state, such as, dominant α wave represents a more relaxed state, and dominant β wave represents a more nervous state. On the other hand, if other physiological sensing element is provided, other physiological signals also can be used to understand the physiological state of the user, such as, the heat rate acquired by the light emitting element and the light receiving element can be the base for obtaining RSA and thus the user's respiratory frequency, for obtaining HRV so as to reveal the ANS activity, and/or for observing the coherence between the heart rate and the respiration, by which it can know that if the user is in a stable physiological state.

Through these pre-observations, the conditions of stimulation can be set in advance, so that the stimulation can be executed in a more effective way. Here, if the brainwaves are observed, then the observation can be, in a continuous period of time or among several time sections, whether the energy distribution within a specific band is stable or not, or whether the peak energies are identical or not. If the observation is about heart rate, then it can be implemented to observe if the heart beat frequency, the respiratory frequency, HRV, and/or the coherence between heart rate and respiration is ranged in a preset range.

Further, if the user is in a physiological state not suitable for stimulation, such as, in an unstable physiological state, it is able to execute the above mentioned biofeedback and/or breathe guiding/breathe training procedure(s) first, and after the user becomes more stable and relaxed, then the resonance stimulation/electrical stimulation can be executed, and thus, the overall effect can be more remarkable. Therefore, there are many possibilities without limitation.

This decision procedure can be implemented to execute on the wearable device or execute by the external device after the physiological signals are transmitted thereto, for example, the physiological signals are wirelessly transmitted to the mobile phone, and the application on the mobile phone does the calculation and decides if the stimulation should be started or which kind of stimulation should be performed.

It shall be noted that as known by one skilled in the arts, the eyeglass structure is one kind of head-mount structure, so that the descriptions above related to the eyeglass structure also can be applied in the devices utilizing the head-mount structure, no matter the device is for acquiring physiological signals or executing stimulation, which all fall within the scope of the present invention.

Another common application is to be used as HMI (Human Machine Interface), for example, by analyzing the acquired EEG signals, the user's intensions can be revealed, or the variations of physiological states can be mapping to different commands. Recently, this kind of HMI is also implemented to cooperate with the biofeedback, and be applied in games, such as, to train the user's concentration through playing games.

Since the sensor, sensing device, and sensing system of the present invention adopt the ear-worn structure and/or the eyeglass structure, it is also suitable for being used as HMI. Under such condition that the acquired physiological signals include EEG signals and heart rate sequence, the commands can be generated by the following possible ways. For example, since the percentage of α waves in brainwaves has significant change along with the action of closing eyes and the action of opening eyes, wherein generally, the percentage of α waves increases significantly as eye closed, this can be the basis to generate the command. Furthermore, when EEG electrodes are located at where EOG signals can be acquired, then the command can be given by the actions of eye blinking, moving/rotating the eye ball. Moreover, since breathing is a controllable physiological activity, and as described above, the respiration can not only influence the heart rate (so called RSA) but also cause fluctuations in a low frequency section of the brain wave, so that in the architecture of the present invention, no matter it is implemented to detect EEG signals or heart rate sequence, both can obtain the variation of respiratory pattern, thereby being the basis to generate the command. For example, the user can intentionally elongate the inhalation period to give command, or also can intentionally increase the depth of respiration to increasing HRV, so as to increase the amplitude of RSA and thus give command. In addition, other physiological signals also can be used as the base, such as, by analyzing EMG signals, it can distinguish if muscles contract or not, and thus, for example, through biting the teeth at the right side or at the left side, commands can be given. Therefore, there are various possibilities without limitation.

Besides, if a movement sensing element, such as, accelerometer, G sensor, gyroscope, magnetic sensor, is further employed, there are more the ways to give commands. For example, all the above mentioned physiological signals can further cooperate the actions of up-down nodding, the head and left-right turning the head, or the actions of the hand, such as, by installing the movement sensing element on the wrist-worn structure or the finger-worn structure to reveal a specific gesture, or a change of the hand's state, so that more combinations can be obtained to give commands, which broadens the application range, such as, it becomes suitable for being used in games. Therefore, there is no limitation.

In the aforesaid, the ear-worn and eyeglass-typed brain activity sensor in accordance with the present invention provides novel contact locations for acquiring EEG signals, namely, the concha wall, the antitragus, the intertragic notch, the tragus, the convex side of the auricle, and/or the V-shaped recess between the auricle and the skull, and also provides a rejecting force in a novel direction, namely, in a direction parallel with the concha floor, for stabilizing the contact between the electrode and the skin at the novel contact locations. Thereby, simply through the wearing action of the sensor, the contact with electrode can be completed and naturally achieve a stable state, which facilitates the acquisition of high quality EEG signals.

Moreover, through such configuration, the applications thereof become more convenient, for example, the physiological stimulations can be performed via the ear-worn structure/the eyeglass structure, and the acquired physiological signals can be the basis to adjust the stimulation. Therefore, not only the wearable type configuration makes the operation more convenient, the effect of stimulation also becomes more remarkable.

1. A wearable physiological activity sensor, for detecting brainwaves from cerebral cortex, comprising:
an in-ear housing, having an EEG electrode mounted thereon;
wherein
the size and the shape of the in-ear housing is configured to enable an engagement with at least a portion of the cymba conchae, the cavum conchae, and/or the intertragic notch of an auricle of an user, and further configured to provide a stable rejecting force at the location of the EEG electrode for achieving a stable contact with the concha wall, the antitragus, the tragus and/or the intertragic notch of the auricle, thereby facilitating an EEG signal acquisition through the EEG electrode.

2. The sensor as claimed in claim 1, further comprising another EEG electrode, wherein said another EEG electrode is configured to locate on one of a group consisting of: the in-ear housing, an extension member connected with the in-ear housing, and another ear-worn structure engaged with another auricle, and said another EEG electrode is configured to contact at least one of a group consisting of: the ear canal, the concha wall, the tragus, the intertragic notch, a V-shaped recess between the auricle and the skull, and the convex side of the auricle.

3. The sensor as claimed in claim 2, further comprising a connection structure for connecting with said another EEG electrode.

4. The sensor as claimed in claim 1, further comprising a light emitting element and a light receiving element mounted on the in-ear housing, for acquiring physiological information of blood from the auricle, wherein the light emitting element and the light receiving element are configured to acquire the physiological information of blood from the tragus and/or the intertragic notch of the auricle.

5. (canceled)

6. (canceled)

7. A wearable physiological activity sensing device, for detecting brainwaves from cerebral cortex, comprising:
a first EEG electrode and a second EEG electrode; and
an ear-worn brain activity sensor, comprising:
an in-ear housing, having the first EEG electrode mounted thereon; and
a physiological signal acquisition circuit, at least partially accommodated in the in-ear housing, for acquiring EEG signals through the first EEG electrode and the second EEG electrode,
wherein
the size and the shape of the in-ear housing is configured to enable an engagement with at least a portion of the cymba conchae, the cavum conchae, and/or the intertragic notch of an auricle of an user, and further configured to provide a stable rejecting force at the location of the first EEG electrode for achieving a stable contact with the tragus, the antitragus and/or the intertragic notch of the auricle; and
during the acquisition of EEG signals, the first EEG electrode is implemented as a reference electrode so as to acquire the EEG signals via reference montage.

8. The device as claimed in claim 7, wherein the second EEG electrode is configured to mount on an eyeglass structure or a head-mount structure, so as to contact at least one of a group consisting of: the region between two eyes, the nasal bridge, the temporal lobe, the occipital lobe, and the frontal lobe, or wherein the second EEG electrode is configured to mount on another ear-worn structure, so as to contact with at least one of a group consisting of: the concha floor of another auricle, a skull area around another auricle, and the skull area around the auricle engaged with the in-ear housing.

9. (canceled)

10. The device as claimed in claim 7, further comprising a light emitting element and a light receiving element mounted on the in-ear housing, for acquiring physiological information of blood from the auricle, wherein the light emitting element and the light receiving element are configured to acquire the physiological information of blood from the tragus and/or the intertragic notch of the auricle.

11. (canceled)

12. (canceled)

13. A wearable physiological activity sensing device, for detecting brainwaves from cerebral cortex, comprising:
two electrodes; and
an ear-worn electrical physiological activity sensor, comprising:
an ear-worn structure, having at least one of the two electrodes mounted thereon,
wherein
the ear-worn structure is configured to mount on at least an auricle of a user, for contacting the at least one electrode thereon with at least one of a group consisting of: concha wall, antitragus, tragus, intertragic notch, the convex side of auricle, and a V-shaped recess between auricle and skull, thereby facilitating an acquisition of electrical physiological signals through the at least one electrode thereon; and
the ear-worn structure is configured to mount on the at least an auricle through at least one of a group consisting of: rejecting force, magnetic attraction, clamping force, and pulling force.

14. The device as claimed in claim 13, wherein the other electrode of the two electrodes is mounted on the ear-worn structure.

15. The device as claimed in claim 13, wherein the electrical physiological signals includes at least one of a group consisting of: EEG signals, ECG signals, EOG signals, EMG signals, and EDA signals.

16. The device as claimed in claim 13, further comprising a light emitting element and a light receiving element mounted on the ear-worn structure, for acquiring physiological information of blood from the auricle and/or the skull around the auricle.

17. (canceled)

18. A wearable physiological activity sensing device, for detecting brainwaves from cerebral cortex, comprising:
a first EEG electrode and a second EEG electrode, for acquiring EEG signals of a user; and
an ear-worn brain activity sensor, comprising:
a first ear-worn structure, which is implemented to be an in-ear housing and have the first EEG electrode mounted thereon; and
a second ear-worn structure, having the second EEG mounted thereon,
wherein
the size and the shape of the in-ear housing is configured to enable an engagement with at least a portion of the cymba conchae, the cavum conchae, and/or the intertragic notch of an auricle of the user, and further configured to provide a stable rejecting force at the location of the first EEG electrode for achieving a stable contact with the concha floor of the auricle; and
the second ear-worn structure is configured to engage with another auricle of the user, so as to contact the second EEG electrode with said another auricle and/or the skull therearound.

19. The device as claimed in claim 18, wherein the second EEG electrode is configured to contact at least one of a group consisting of: the ear canal, the concha wall, the tragus, the antitragus, the intertragic notch, the concha floor, the convex side of auricle, a V-shaped recess between auricle and skull, and the skull.

20-145. (canceled)

146. The sensor as claimed in claim 1, further comprising a covering member, for covering at least a portion of the in-ear housing, wherein the covering member is configured to have an electrode mounted on a surface thereof and electrically connected to the EEG electrode, thereby replacing the EEG electrode to acquire the EEG signals, and the engagement with the auricle is further achieved by the cover member.

147. The sensor as claimed in claim 2, wherein at least one of the EEG electrode and said another EEG electrode is implemented to have a contact assurance structure.

148. The device as claimed in claim 7, further comprising a covering member, for covering at least a portion of the in-ear housing, wherein the covering member is configured to have an electrode mounted on a surface thereof and electrically connected to the first EEG electrode, thereby replacing the first EEG electrode to acquire the EEG signals, and the engagement with the auricle is further achieved by the cover member.

149. The device as claimed in claim 7, wherein at least one of the first EEG electrode and the second EEG electrode is implemented to have a contact assurance structure.

150. The device as claimed in claim 13, wherein the ear-worn structure is implemented to be an ear-hooking structure having a front ear member and an extension member, and the at least one electrode is mounted on the front ear member and/or the extension member for achieving the contact with the skin.
